(19) United States
(12) Patent Application Publication
(10) Pub. No.: US 2024/0271099 A1
(43) Pub. Date: Aug. 15, 2024

(54) METHOD FOR GENERATING HIGHLY FUNCTIONAL HEPATOCYTES BY DIFFERENTIATING HEPATOBLASTS

(71) Applicants: INSTITUT NATIONAL DE LA SANTÉ ET DE LA RECHERCHE MÉDICALE, Paris (FR); UNIVERSITÉ PARIS-SACLAY, Gif-sur-Yvette (FR)

(72) Inventors: Anne DUBART-KUPPERSCHMITT, Villejuif (FR); Eleanor LUCE, Villejuif (FR); Antonietta MESSINA, Villejuif (FR)

(21) Appl. No.: 18/567,830

(22) PCT Filed: Jun. 3, 2022
(86) PCT No.: PCT/EP2022/065167

§ 371 Date: Dec. 7, 2023

(30) Foreign Application Priority Data
Jun. 7, 2021 (EP) 21305772.2

(51) Int. Cl. C12N 5/071; A61K 35/407

(52) U.S. Cl. CPC C12N 5/067; A61K 35/407; C12N 2500/38; C12N 2501/12; C12N 2501/15; C12N 2501/237; C12N 2501/39; C12N 2503/02

(57) ABSTRACT

Human pluripotent stem cells (hPSCs) are a concrete source of hepatic cells for regenerative medicine applications and are largely contributing to the study of liver diseases, toxicity, and drug efficacy. However, hP SC-derived hepatocyte-like cells possess morphological and functional features typical of foetal hepatocytes rather than post-natal or adult hepatocytes. By self-assembling hepatic progenitors into spheroids and by refining the maturation step of their differentiation protocol, the inventors aim at generating hPSC-derived hepatocyte-like cells with an improved maturation degree, showing morphological and functional features of adult hepatocytes. More particularly, they adjusted the morphogen cocktail used for the maturation step by the regular administration of vitamin Kl, a daily regulation of glucocorticoid supply, and a progressive decrease of Oncostatin M (OSM) supply in the last days. They demonstrated that the hepatocytes produced with their protocol have reached the highly functional ability of primary human hepatocytes, an improved maturation stage compared to previously reported data on hPSC-derived hepatocytes. Thus the invention relates to a new method for improving the differentiation of hepatoblasts into hepatocytes.

### FIELD OF THE INVENTION

The invention relates to a method for generating highly functional hepatocytes by differentiating hepatoblasts.

### BACKGROUND OF THE INVENTION

Human induced pluripotent stem cells (hiPSCs) are a concrete source of hepatic cells for regenerative medicine applications which are largely contributing to the study of liver diseases, toxicity, and efficacy of drugs [1-8]. Indeed, hiPSC-derived hepatocytes (iHeps) are able to perform a variety of diverse functions, including protein synthesis, energy metabolism, detoxification of endogenous and exogenous substances, as well as bile secretion [9]. However, it has been demonstrated that the generated iHeps exhibit an immature hepatic phenotype rather than the post-natal or the adult one, particularly when they are generated in two-dimensional (2D) cell cultures. Notably, cells persistently express foetal markers like alpha fetoprotein (AFP) and are poor in reproducing key mature hepatocyte functions such as the activity of many detoxification enzymes, reaching 0.1% of the primary human hepatocytes (PHHs)' ability [10-12]. Such an important limitation can be overcome through in vivo transplantation. Stem cell-derived hepatocyte-like cells (HLCs) engrafted in animal models showed to enable the host liver functional rescue and to induce the final stage maturation of the transplanted cells [13-15]. However, for specific tissue engineering applications such as the development of a bioartificial liver device (BAL) or pharmaceutical applications, the cell maturation must necessarily be achieved in vitro (since their transplantation is not foreseen). Multiple differentiation protocols have been proposed in the last years. Some have attempted to optimize iHep maturation by i) supplying inhibitors of the NOTCH or TGFβ pathways like compound E [10] and SB431542 [10,11], in combination with hepatocyte growth factor (HGF), glucocorticoids, like dexamethasone (DEX), and oncostatin M (OSM) [16,17]; co-culturing iHeps with non-parenchymal cells, such as stellate cells or endothelial cells [18-21]; providing physical and mechanical support using extracellular matrices or scaffolds [22-26]. Furthermore, to better represent physiological conditions, 3D culture systems have been developed and liver organoids generated [27,28]. Indeed, these new approaches provided suitable alternatives to generate hepatocytes showing higher activity and long-term functions in vitro, and have proven useful in developmental and toxicological studies, drug discovery, disease modelling as well as bioprinting and biofabrication methods [4,29,30]. Nevertheless, fully mature hepatocytes have not yet been produced. Indeed, it has been highlighted that none of the past studies have resulted in producing fully functional hepatocytes. Transcriptomic analysis allowed to compare iHep expression patterns to hepatic cell lines (HepaRG, HepG2 cells) and PHHs revealing 80% similarity between iHeps and PHHs. However, differences in cell sources, data, and analytical procedures led to different and controversial results [32-35].

Recently the inventors demonstrated that by refining the last step of the differentiation protocol, a good degree of maturation can be obtained in 2D systems [12]. Cells obtained closely resembled PHHs in morphology and expressed most of the specific hepatic markers (asialoglycoprotein receptor (ASGR), albumin (ALB), connexion 32 (CX32)), as well as epithelial polarity (zonula occludens 1 (ZO-1), claudin-1 (CLDN1), multidrug resistance-associated protein 2 (MRP-2)) and cytochrome P450 activity (CYP3A4, CYP2A6, and CYP2D6).

Herein the inventors optimized the culture conditions by self-assembling hiPSC-derived liver progenitors in spheroids and proceeded with the gradual then complete removal of the oncostatin M (OSM). Cells were then widely characterized in terms of morphology, protein expression, and through multiple functional assays. Data recorded show that iHeps have reached an improved maturation stage compared to previously reported data and bibliography [33-39].

### SUMMARY OF THE INVENTION

The invention relates to a method for improving the differentiation of hepatoblasts into hepatocytes. More particularly, the present invention relates to a method for improving the differentiation of hepatoblasts into highly functional hepatocytes, i.e showing functional features of adult hepatocytes with no remaining foetal gene expression.

In particular, the present invention is defined by the claims.

### DETAILED DESCRIPTION OF THE INVENTION

By self-assembling hepatic progenitors into spheroids and by refining the maturation step of their differentiation protocol, the inventors aim at generating iHeps with an improved maturation degree, showing morphological and functional features of adult hepatocytes. More particularly, they finely adjusted the additive cocktail used for the maturation step by the regular administration of vitamin K1, a daily regulation of glucocorticoid supply, and a progressive suppression of OSM supply in the last days. They demonstrated that the iHeps produced with their protocol have reached the functional ability of PHHs, an improved maturation stage compared to previously reported data on iHeps [19-25].

### 1) Differentiation Protocol

Accordingly, the present invention relates to a method for improving the differentiation of hepatoblasts into hepatocytes comprising culturing said hepatoblasts in hepatocyte medium supplemented with hepatocyte growth factor (HGF), glucocorticoid, and oncostatin M wherein the medium supplemented is used and refreshed every day and wherein:
i) from the fourth, fifth, sixth, or seventh day of culture until the end of the culture, vitamin K is added as a supplement in the medium
ii) from the eighth or ninth day of culture until the end of the culture, the glucocorticoid is administered to the culture so that its concentration decreased by half and reverted back to its initial concentration every other day,
iii) from the ninth, tenth or eleventh day of culture until the end of the culture, oncostatin M is administered in the medium at a concentration decreased by half every other day.
iv) from the ninth, tenth or eleventh day of culture until the end of the culture, notch inhibitor and TGF-β receptor are added to the culture.

As used herein, the term “hepatocyte medium” refers to a highly reproducible maintenance serum-free medium for primary hepatocytes from all species. Hepatocyte medium includes a medium suitable to the culture of primary hepatocytes, namely optimized for hepatocyte culture and compatible with all hepatocyte species. Medium suitable for the culture of hepatocytes are commercially available and include, but are not limited to HBM™ Hepatocyte Basal Medium and HCM™ Hepatocyte Culture Medium (LONZA, ref. 11685450).

In some embodiment, the hepatocyte medium is complete medium HCM™.

In some embodiment, the hepatocyte medium is complete medium HCM™ without hydrocortisone.

In some embodiment, the hepatocyte medium comprises or consists of the following constitution (HPM medium):

HPM medium

| Components | Concentration | |
|---|---|---|
| William's E medium | 50% | |
| Ham F12 medium | 50% | |
| Penicillin and/or Streptomycin | 1% | |
| L-glutamine | 1% | |
| Linoleic acid-Albumin | 1.10⁻⁵M | |
| Triiodo-L-thyronine | 5.10⁻⁸M | |
| Insulin | 0.07 | IU |
| Vitamin C | 6.10⁻⁴M | |
| Human apo-transferrin | 6.10⁻⁴M | |
| Sodium Pyruvate | 1 | mM |
| Selenic acid | 0.5 | nM |

In some embodiment, the hepatocyte medium comprises or consists of the following constitution (iHepM medium):

iHepM medium

| Components | Concentration |
|---|---|
| William's E medium | 100% |
| Penicillin and/or Streptomycin | 1% |
| L-glutamine | 1% |
| Insulin-Transferrin-Selenium (ITS-G) | 1% |
| HEPES | 10 mM |

The hepatocyte medium supplemented according to the present invention may be prepared from individual separate ingredients, commercially available as culture grade powders, solutions, suspensions, or emulsions. In some embodiment, the hepatocyte medium supplemented according to the present invention is prepared by adding step-wise each supplement component in the hepatocyte medium.

As used herein the term “hepatoblasts” has its general meaning in the art and refers to hepatic progenitor cells that expand and give rise to either hepatocytes or cholangiocytes during liver development. They refer to cells that are capable of expressing characteristic biochemical markers, including but not limited to Alpha-fetoprotein (AFP), Cytokeratin 19 (CK19), EP-CAM, and Hepatocyte nuclear factor 4 alpha (HNF4alpha). Hepatoblasts may be derived from pluripotent stem cells such as embryonic stem cells (ESCs) and induced pluripotent stem cells (iPSCs).

In some embodiment, the hepatoblasts are pluripotent stem cell-derived hepatoblasts.

In some embodiment, the hepatoblasts are human pluripotent stem cell-derived hepatoblasts.

In some embodiment, the hepatoblasts are human embryonic stem cell-derived hepatoblasts or human induced pluripotent stem cell-derived hepatoblasts.

As used herein, the term “embryonic stem cells” refers to embryonic cells, which are pluripotent stem cells capable of differentiating into cells of all three embryonic germ layers (i.e., endoderm, ectoderm, and mesoderm), and proliferating in an undifferentiated state. Such cells may comprise cells that are obtained from the embryonic tissue formed after gestation (e.g., blastocyst) before implantation of the embryo (i.e., a pre-implantation blastocyst). The embryonic stem cells may be obtained using well-known cell-culture methods (see U.S. Pat. No. 5,843,780). For example, human embryonic stem cells can be isolated from human blastocysts. Human blastocysts are typically obtained from in vitro fertilization-derived (IVF) embryos. Commercially available stem cells may also be used. Human ES cells can be purchased from the NIH human embryonic stem cells registry (http://escr.nih.gov).

As used herein the term “induced pluripotent stem cells (iPSCs)” has its general meaning in the art and refers to a type of pluripotent stem cells that can be generated directly from a somatic cell. Human induced pluripotent stem cells (hiPSCs) are a concrete source of hepatic cells for regenerative medicine applications and are largely contributing to the study of liver diseases, toxicity, and drug screening. Exemplary protocols for the generation of iPSCs, and more particularly to human induced pluripotent stem cells (hiPSCs), are known to those of skill in the art. Methods for generating induced pluripotent stem cells based on expression vectors encoding reprogramming factors have been described in the art, see for example WO2007/69666, EP2096169-A1, or WO2010/042490. Exemplary protocols for differentiating hepatoblasts from induced pluripotent stem cells (iPSCs), and more particularly from human pluripotent stem cells (hiPSCs) are known to those of skill in the art and are briefly described in various publications [36-38].

In some embodiment, the method for improving the differentiation of hepatoblasts into hepatocytes is performed in 2D or 3D culture.

As used herein, the term “2D culture” has its general meaning in the art and refers to cultures of cells on flat plastic dishes. Indeed, in adherent 2D cultures, cells grow as a monolayer in a culture flask or a petri dish, attached to a plastic surface that could be coated to promote cell adhesion and proliferation. The advantages of 2D cultures are associated with simple and low-cost maintenance of the cell culture and with the performance of imaging and functional tests. Unfortunately, adherent cultures also have numerous disadvantages. After isolation from the tissue and transfer to the 2D conditions, the cell morphology is altered, as well as the cell division can be affected [39].

In some embodiment, the method for improving the differentiation of hepatoblasts into hepatocytes is performed in 2D culture, wherein the hepatoblasts were cultured on multiwell plates coated with a homemade coating solution constituted of 1% w/v fibronectin, 3% w/v calf skin collagen type I, and 10% w/v bovine serum albumin (BSA) [12].

As used herein, the term “3D culture” has its general meaning in the art and refers to an artificially created environment in which biological cells are permitted to grow or interact with their surroundings in all three dimensions. Unlike 2D cell culture, a 3D cell culture allows cells to grow in vitro in all directions, similar to how they would do in vivo. Due to the method of preparation, 3D cultures can be divided into i) suspension cultures on non-adherent plates; ii) cultures in concentrated medium or in gel-like substances enriched in extracellular matrix proteins, and iii) cultures on a scaffold [39]. The concept of 3D spheres is based on the creation of spheroid structures in which cells self-organize. According to the invention, the spheroid structures have been produced by i) creating poly(dimethylsiloxane) (PDMS) moulds as previously reported [40] constituted of 63 μ-cylinders of 1 mm diameter and depth, ii) creating non-adherent uwells by placing said PDMS moulds in 2% liquid agarose solution into culture plates wherein the PDMS moulds are placed upside down.

By triggering the self-assembling of hepatic progenitors into spheroids and by refining the maturation step of their differentiation protocol, the inventors produced hiPSC-derived hepatocytes (iHeps) with an improved maturation degree compared to those obtained using previously reported protocols.

Thus, in some embodiment, the method for improving the differentiation of hepatoblasts into hepatocytes is performed in 3D culture.

In a particular embodiment, the hepatoblasts were seeded to obtain spheroids with a diameter of 50-500 μm.

In a particular embodiment, the hepatoblasts were seeded to obtain spheroids with a diameter of 50, 100, 150, 200, 250, 300, 350, 400, 450, or 500 μm.

As used herein the term “hepatocyte” has its general meaning in the art and refers to the major parenchymal cells of the liver that represent up to 70-85% of the liver mass. Hepatocytes are highly differentiated cells that carry out most of the hepatic functions, which pertain notably to metabolism, detoxification, and systemic homeostasis. Hepatocytes have a unique complex polarization essential to some of their functions with basolateral membranes facing the sinusoids, and apical membranes forming bile canaliculi. Indeed, cell signaling, membrane trafficking, protein secretion, and bile transport are allowed because of this peculiar polarization. Hepatocytes include human hepatocytes and other mammalian hepatocytes such as non-human primate hepatocytes, pork hepatocytes, or mouse hepatocytes. Hepatocytes differentiated from human pluripotent stem cells such as hiPSCs or hESCs, possess morphological and functional features typical of foetal hepatocytes rather than post-natal or adult hepatocytes [14]. Notably, hiPSC-derived hepatocytes (iHeps) persistently express foetal markers like alpha-fetoprotein (AFP) and lack or poorly provide key mature hepatocyte functions, such as the activity of many detoxification enzymes (CYP2A6, CYP3A4), which represent about 0.1% of the primary human hepatocyte (PHH) ability. Moreover, hiPSC-derived hepatocytes (iHeps) often lack the unique polarization of PHHs. Such a disadvantage is undoubtedly largely due to the fact that most hepatocyte differentiation protocols were developed in two-dimensional culture systems. Primary hepatocytes are also known to lose their unique polarisation when grown as monolayer. Indeed, their polarity consists of a complex organization of structural and functional components (such as tight junction proteins and apical membrane transporters) which form bile canaliculi organising a 3D network. In 2D cultures, hepatocytes revert to a simple epithelial phenotype losing such a complex polarization.

In some embodiment, the hepatocytes differentiated by the method of the invention exhibit highly functional features of adult hepatocytes.

In some embodiment, the hepatocytes differentiated by the method of the invention do not exhibit foetal hepatocyte gene expression.

In some embodiment, the hepatocytes differentiated by the method of the invention exhibit highly functional features of adult hepatocytes and do not exhibit foetal hepatocyte gene expression.

In some embodiment, the hepatocytes differentiated by the method of the invention exhibit the tested hepatic functions similar or superior to adult hepatocytes (see Results).

In some embodiment, the hepatocytes differentiated by the method of the invention exhibit a polarization similar to PHHs. In some embodiment, the hepatocytes differentiated by the method of the invention exhibit cytochrome P450 activity.

In some embodiment, the hepatocytes differentiated by the method of the invention expressed the hepatocyte markers: asialoglycoprotein receptor (ASGR), albumin (ALB), cytochrome CYP3A4, connexion 32 (CX32), zonula occludens-1 (ZO-1), claudin-1 (CLDN1) and multidrug resistance-associated protein 2 (MRP-2).

In some embodiment, the hepatocytes differentiated by the method of the invention expressed the hepatocyte markers: asialoglycoprotein receptor (ASGR), albumin (ALB), cytochrome CYP3A4, connexion 32 (CX32), zonula occludens-1 (ZO-1), claudin-1 (CLDN1) and multidrug resistance-associated protein 2 (MRP-2) and exhibit hepatic cytochrome P450 activity, uridine diphosphate glucuronosyltransferase 1A1 (UGT1A1) activity and alcohol dehydrogenase (ADH) activity.

According to the invention, the term “hepatic cytochrome P450 activity” refers to the activity of CYP3A4, CYP2A6, CYP1A1, CYP1A2, CYP2B6 and CYP2D6. Cytochrome P450, such as CYP3A4, CYP2A6, CYP1A1, CYP1A2, CYP2B6 and CYP2D6 are oxidative metabolic enzymes that play critical roles in the biotransformation of endogenous compounds and xenobiotics.

In some embodiment, the hepatocytes differentiated by the method of the invention can exhibit bile acids (BA) production, such as cholic acid (CA), chenodeoxycholic acid (CDCA) and glycocholic acid (GCA), similar to PHHs.

In some embodiment, the hepatocytes differentiated by the method of the invention form bile canaliculi organising a 3D network (see FIG. 6C and 6D).

Herein, the inventors demonstrated that their optimized protocol allows to maintain the culture of hiPSC-derived hepatocytes during at least 17 days in 2D culture and at least 34 days in 3D culture.

Thus, the method of the invention may be used to maintain a culture of hepatoblast-derived hepatocytes or hepatocytes.

In some embodiment, the 2D culture of hepatoblast-derived hepatocytes of the invention is maintained for at least 17 days. In some embodiment, the 3D culture of hepatoblast-induced hepatocytes of the invention is maintained for at least 34 days.

As used herein, the term “hepatocyte growth factor” (HGF), also known as “scatter factor” has its general meaning in the art and refers to a paracrine cellular growth, motility and morphogenic factor. HGF is among a group of factors possessing an angiogenic ability that are described as heparin-binding growth factors. HGF is secreted by fibroblasts and is mitogenic for epithelial and endothelial cells and also melanocytes, but does not affect fibroblasts. HGF possesses the ability to promote cell proliferation, resistance to apoptosis, and induces cell motility or invasion. The endogenous human HGF protein has an aminoacid sequence as shown in Uniprot Accession number P14210.

In some embodiment, HGF is added in the medium at a concentration ranging from 10 ng/mL to 100 ng/mL. In some embodiment, HGF is added in the medium at a concentration of 10, 15, 20, 25, 30, 35, 40, 45, 50, 55, 60, 65, 70, 75, 80, 85, 90, 95 or 100 ng/ml. In some embodiment, HGF is added in the medium at a concentration of 20 ng/ml.

As used herein, the term “glucocorticoid” has its general meaning in the art and refers to a class of corticosteroids that bind to the glucocorticoid receptor present in almost every vertebrate animal cell. Glucocorticoid receptors are transacting transcription factors that can modulate gene expression by binding to DNA sites. Glucocorticoids play important roles in development and homeostasis. It had been shown that glucocorticoid supplements, in particular through dexamethasone, highly affect the maturation of foetal and neonatal hepatocytes towards adult hepatocytes contributing to the up-regulation of connexin 32 and connexin 26 transcript levels [41,42].

According to the invention, the term “glucocorticoid” includes but is not limited to cortisol, cortisone, prednisone, prednisolone, methylprednisolone, dexamethasone, betamethasone, hydrocortisone, triamcinolone, fludrocortisone, deoxycorticosterone, aldosterone and beclomethasone.

In a particular embodiment, glucocorticoid is added in the medium at a concentration ranging from 0.05 nM to 0.15 nM, and more particularly at a concentration of 0.1 nM. In some embodiment, glucocorticoid is added in the medium at a concentration of 0.1 nM and ii) from the ninth day of culture it is administered in the medium so that its concentration decreased by 0.05 nM and reversed back to 0.1 nM every 24 h.

In a particular embodiment, the glucocorticoid added in the medium is dexamethasone.

As used herein, the term “dexamethasone” (DEX) has its general meaning in the art and refers to a synthetic glucocorticoid, similar to the natural glucocorticoid hydrocortisone. It is an anti-inflammatory glucocorticoid with a range of effects on cell survival, cell signaling and gene expression. Dexamethasone is an important regulator of cellular proliferation and differentiation.

As used herein, the term “oncostatin M” (OSM) has its general meaning in the art and refers to a multifunctional cytokine that belongs to the interleukin-6 (IL-6) subfamily. This cytokine acts on a wide variety of cells and elicits diverse overlapping biological responses such as growth regulation, differentiation, gene expression, and cell survival. Oncostatin M was demonstrated to strongly enhance the differentiation of fotal hepatocytes [27].

In some embodiment, OSM is added in the medium at a concentration ranging from 5 ng/ml to 30 ng/mL. In some embodiment, OSM is added in the medium at a concentration of 5, 6, 7, 8, 9, 10, 11, 12, 13, 14, 15, 16, 17, 18, 19, 20, 21, 22, 23, 24, 25, 26, 27, 28, 29 or 30 ng/ml. In some embodiment, OSM is added in the medium at a concentration of 20 ng/ml.

As used herein, the term “vitamin K” has its general meaning in the art and refers to a group of fat-soluble vitamins acting as indispensable cofactors in the post-translational γ-carboxylation of glutamic acid residues of coagulation-associated proteins such as factors II (prothrombin), VII, IX and X [43,44]. It proved, in animal models, to be accountable for the regulation of the signalling pathways of inflammation (NF-κB) processes, glucose metabolism (SIRT1/AMPK/PI3K/PTEN/GLUT2/GK/G6Pase), and lipid oxidation (PPARα/CPT1A) [45,46]. According to the invention, the term “vitamin K” includes but is not limited to vitamin K1 and vitamin K2. As used herein, the term “vitamin K1” has its general meaning in the art and refers to the major dietary source and primary circulating form of vitamin K.

In a particular embodiment, the vitamin K added in the medium i) from the seventh day of culture is vitamin K1 or vitamin K2.

In a particular embodiment, vitamin K1 is added in the medium i) from the seventh day of culture at a concentration ranging from 5 ng/ml to 1 mg/mL, and more particularly at a concentration of 10 ng/ml.

In a particular embodiment, vitamin K2 is added in the medium i) from the seventh day of culture at a concentration ranging from 1 nM to 7 nM.

As used herein, the term “Notch inhibitor” has its general meaning in the art and refers to a compound inhibiting the Notch signalling pathway. The Notch signalling pathway is a highly conserved cell signalling system present in most animals. Mammals possess four different Notch receptors, referred to as Notch1, Notch2, Notch3, and Notch4. Notch-signalling pathway is evolutionarily conserved and plays a major role in embryonic vascular development and angiogenesis. Notch-signalling pathway is important for cell-cell communication, which involves gene regulation mechanisms that control multiple cell differentiation processes during embryonic and adult life. It has been shown that one method of effectively blocking Notch activity is preventing its cleavage at the cell surface with y-secretase inhibitors [72]. As used herein, the term “gamma-secretase inhibitor” for “γ-secretase inhibitor” has its general meaning in the art and refers to compound inhibiting γ-secretase, a multi-subunit protease complex. According to the invention, the term γ-secretase inhibitor includes but is not limited to γ-Secretase Inhibitor XXI (also known as compound E, referenced under CAS number: 209986-17-4), Compound W (referenced under CAS number: 173550-33-9), γ-Secretase Inhibitor IX (also known as DAPT, referenced under CAS number: 208255-80-5), γ-Secretase Inhibitor I, γ-Secretase Inhibitor X (LY-685458 referenced under CAS number: 292632-98-5), γ-Secretase Inhibitor XX (Dibenzapine referenced under CAS number: 209984-56-5), RO-4929097 (referenced under CAS number: 847925-91-1), MRK-003 (referenced under CAS number: 623165-93-5), MRK-0752 (referenced under CAS number: 471905-41-6), LY411575 (referenced under CAS number: 209984-57-6), nirogacestast (also known as PF-03084014, referenced under CAS number: 1290543-63-3), begacestat (also known as GSI-953, referenced under CAS number: 769169-27-9), avagacestat (also known as BMS-708163, referenced under CAS number: 1146699-66-2), ELN-475516 (referenced under CAS number: 926658-65-3), LY-450139 (referenced under CAS number: 425386-60-3), JLK6 (referenced under CAS number: 209984-56-5), Flurizan (referenced under CAS number: 51543-40-9), and MRK-560 (referenced under CAS number: 677772-84-8).

In a particular embodiment, i) from the ninth day of culture, the Notch inhibitor is added in the medium at a concentration ranging from 0.3 nM to 0.7 nM, and more particularly at a concentration of 0.5 nM.

In some embodiment, the Notch inhibitor added in the medium i) from ninth day of culture is γ-secretase inhibitor. In some embodiment, the notch inhibitor added in the medium i) from ninth day of culture is compound E.

As used herein, the term “TGF-β Receptor inhibitor” has its general meaning in the art and refers to a compound inhibiting the TGF-β Receptor. Transforming growth factor beta (TGFB) receptors are single-pass serine/threonine kinase receptors that belong to TGFβ receptor family. It has been shown that members of the transforming growth factor (TGF)-β superfamily play important roles during the differentiation of vascular progenitor cells derived from mouse embryonic stem cells (ESCs). Synthetic molecule inhibiting TGF-β Receptor inhibitor facilitated proliferation and sheet formation of ESC-derived endothelial cells [73]. According to the invention, TGF-β Receptor inhibitor includes but are not limited to SB431542 (referenced under CAS number: 301836-41-9), LDN-193189 (referenced under CAS number: 1062368-24-4), Galunisertibe (also known as LY2157299, referenced under CAS number: 700874-72-2), LY2109761 (referenced under CAS number: 700874-71-1), SB525334 (referenced under CAS number: 356559-20-1), SB505124 (referenced under CAS number: 694433-59-5), GW788388, (referenced under CAS number: 452342-67-5), LY364947 (also known as HTS 466284, referenced under CAS number: 396129-53-6), RepSox (referenced under CAS number: 446859-33-2), LDN-193189 2HCL (referenced under CAS number: 1435934-00-1), K02288 (referenced under CAS number: 1431985-92-0), BIBF-0775 (referenced under CAS number 334951-90-5), TP0427736 HCl (referenced under CAS number: 864374-00-5), A-83-01 (referenced under CAS number: 909910-43-6), LDN-214117 (referenced under CAS number: 1627503-67-6), SD-208 (referenced under CAS number: 627536-09-8), vactosertib (referenced under CAS number: 1352608-82-2), R-268712 (referenced under CAS number: 879487-87-3) and ML347 (referenced under CAS number: 1062368-49-3).

In some embodiment, i) from the ninth day of culture, TGF-β receptor inhibitor is added in the medium at a concentration ranging from 3 nM to 7 nM. In some embodiment, i) from the ninth day of culture, TGF-β receptor inhibitor is added in the medium at a concentration of 5 nM.

In some embodiment, the TGF-β Receptor inhibitor is SB431542.

### 2) Application of the Method of the Invention

The present invention provides a method for improving the differentiation of hepatoblasts into hepatocytes, wherein the hepatocytes obtained reached an increased maturation degree compared to those obtained in previously reported protocols. Indeed, the inventors show that hepatocytes differentiated by the method of the invention possess highly functional features typical of mature adult hepatocytes.

The methods and the uses of hepatocytes obtained by the method encompassed by the instant application provide potential numerous applications, such as:
providing an in vitro cellular model to study liver diseases.
providing a particularly well-suited model for the study of infectious disease.
providing a cellular microenvironment for liver tissue engineering, to produce bio-artificial liver devices.
providing a cellular microenvironment for drug screening.
providing a cellular microenvironment for toxicological studies.
providing a cellular microenvironment for liver tissue engineering.
providing a cellular microenvironment for cell-based therapy and regenerative medicine.
providing a cellular microenvironment to investigate cell-based therapy and regenerative medicine.

As used herein, the term “liver disease” has its general meaning in the art and refers to any disturbance of liver function that causes illness. The liver is responsible for many critical functions within the body and should it become diseased or injured, the loss of those functions can cause significant damage to the body. Liver disease is also referred to as a hepatic disease. Many diseases and conditions can affect the liver, for example, certain drugs like an excessive amount of acetaminophen, and acetaminophen combination medications like Vicodin and Norco, as well as statins, cirrhosis, alcohol abuse, hepatitis A, B, C, D, and E, infectious mononucleosis (Epstein Barr virus), non-alcoholic fatty liver disease (NASH), and iron overload (hemochromatosis).

As used herein, the term “infectious disease” has its general meaning in the art and refers to disorders caused by a pathogen such as a bacterium, a virus, a protozoan, a prion, a viroid, or a fungus.

As used herein, the term “bio-artificial liver device” (BAL) has its general meaning in the art and refers to an artificial extracorporeal liver support (ELS) system that includes hepatocytes into a bioreactor operating alongside the purification circuits used in artificial ELS systems. These devices are used for treating an individual who is suffering from a liver disease such as acute liver failure (ALF) or acute-on-chronic liver failure (ACLF).

Thus, the present invention relates to a method for obtaining a population of hepatocytes comprising the step of differentiating hepatoblasts into hepatocytes according to the method of the invention.

The present invention also relates to the population of hepatocytes obtainable by the method of the invention for use in therapy.

In some embodiment, the therapy is cell-based therapy or regenerative medicine.

As used herein, the term “cell therapy” has its general meaning in the art and refers to transplanting cells in order to restore tissue or organ function.

As used herein, the term “regenerative medicine” has its general meaning in the art and refers to a process of replacing, engineering or regenerating human or animal cells, tissues or organs to restore or establish normal function.

The present invention relates to the population of hepatocytes obtainable by the method of the invention for use in the treatment of liver disease.

In other words, the invention relates to a method for treating liver diseases comprising administering the population of hepatocytes obtainable by the methods of the invention.

In the context of the invention, the terms “treating” or “treatment”, as used herein, refer to a method that aims at delaying or preventing the onset of a pathology, at reversing, alleviating, inhibiting, slowing down or stopping the progression, aggravation or deterioration of the symptoms of the pathology, at bringing about amelioration of the symptoms of the pathology, and/or at curing the pathology.

In some embodiment, the invention relates to the population of hepatocytes obtained by the method of the invention for use in bio-artificial liver devices.

Animal testing plays a crucial role in predicting pharmacokinetics and toxicology as a preclinical test in drug discovery. Early determination of metabolic pathways and the rates of metabolism in different test species may provide explanations for severity and reversibility of toxicity, the relationship to the mode and time of exposure as well as the determination of the dose ranges. However, erroneous pharmacokinetic and toxicological predictions due to differences between the human species and laboratory animals have led to the abandonment of some candidate compounds before clinical trials.

Thus, in some embodiment, the invention relates to the population of hepatocytes obtained by the method of the invention for drug screening, and in particular in pre-clinical drugs screening.

The population of hepatocytes obtained by the method of the invention may be used to complement or replace toxicological studies in animal models.

Thus, in some embodiment, the invention relates to the population of hepatocytes obtained by the method of the invention for use in toxicological studies.

In another aspect, the population of hepatocytes obtained by the method of the invention from pluripotent stem cells derived from healthy or diseased patients may also be used advantageously for screening applications in the pharmaceutical industry. Such screening tests can be used to search for new drugs with clinical applications or for toxicology tests.

Accordingly, the invention provides an in vitro method of screening for a compound useful in the treatment of a liver disease comprising the steps of:
(a) contacting a population of hepatocytes produced by a method of the invention from healthy or diseased patients with a test compound, and;
(b) determining the effect of the test compound on said hepatocytes.

In one embodiment, the test compound may be selected from the group consisting of peptides, proteins, peptidomimetics, small organic molecules, aptamers or nucleic acids. For example, the test compound according to the invention may be selected from a library of compounds previously synthesised, or a library of compounds for which the structure is determined in a database, or from a library of compounds that have been synthesised de novo.

In a particular embodiment, the test compound may be selected from small organic molecules. As used herein, the term “small organic molecule” refers to a molecule of size comparable to those organic molecules generally used in pharmaceuticals.

In particular, the population of hepatocytes obtained by the methods of the invention, namely differentiating hepatocytes from pluripotent stem cells derived from healthy or diseased patients, may also be used advantageously to produce in vitro cellular models to study liver diseases, and in particular, inherited genetic liver diseases.

The population of hepatocytes obtained by the method of the invention from pluripotent stem cells derived from healthy or diseased patients may also be used advantageously to study the pathophysiology of liver diseases.

### 3) Kit for Performing the Method According to the Invention

In another aspect, the invention relates to a kit for performing the methods of the present invention, wherein said kit comprises hepatocyte medium, oncostatin M, hepatocyte growth factor, glucocorticoid, vitamin K, Notch inhibitor and TGF-β receptor inhibitor.

More particularly, the kit comprising:
(a) hepatocyte medium, oncostatin M, hepatocyte growth factor, glucocorticoid, vitamin K, Notch inhibitor and TGF-β receptor inhibitor, and
(b) instructions for use.

In some embodiment, the kit comprising:
(a) hepatocyte medium, dexamethasone, oncostatin M, hepatocyte growth factor, vitamin K, compound E and SB431542, and
(b) instructions for use.

The invention will be further illustrated by the following example. However, the example should not be interpreted in any way as limiting the scope of the present invention

### FIGURES

FIG. 1: Immunofluorescence staining of proteins specific to hiPSCs, endoderm cells (DE) and hepatoblasts (iHBs). Scale bar of day 0—hiPSCs=500 μm; Scale bar of day 5—DE and day 11—iHBs=100 μm.

FIG. 2: hiPSC differentiation into hepatocytes by self-assembling process.

A. Expression of hepatic specific markers (RT-PCR) in 3D iHeps. B. Gene expression of AFP, ALB, CYP3A7 and CYP3A4 analysed by RT-PCR in 3D iHeps and PHH spheroids. C. AFP, ALB, and CYP3A4 gene expression in 3D iHeps over time (black bars). PHHs have been used as control (white bars). Quantification is relative to the expression level in foetal human hepatocytes (Gestation Stage 20 weeks). Histograms represent mean±SD (n=16). *** indicates p<0.001; ** indicates p<0.01; * indicates p<0.05

FIG. 3: Enzyme-linked immunosorbent assay (ELISA) on iHeps.

Enzyme-linked immunosorbent assay (ELISA) for A. AFP and B. ALB secretion in 2D iHeps generated with our previously published protocol (white bars), 2D iHeps generated with our new protocol (hatched bars) and 3D iHeps (black bars). The data are representative of sixteen independent experiments and samples were analysed in triplicates. C. ELISA of ALB secretion in 3D iHeps along the time. DNA contents were used to quantify cell number per sample so that quantification assays' results are shown as μg/ml/10⁶/24 h if not otherwise specified. D. ELISA quantification of AFP and ALB secretion of 3D iHeps (black bars) and PHH spheroids (white bars). *** indicates p<0.001; ** indicates p<0.01; * indicates p<0.05

FIG. 4: Activity of phase I and II metabolisms of iHeps. Luminescence data for A. CYP1A1/2 and B. CYP3A4 isoforms; activities in 2D and 3D iHeps are shown as the comparison between rifampicin non-induced and induced iHep cultures at day 28. Six independent experiments were performed for each condition and samples were analysed in triplicates. C. CYP1A1, CYP1A2, CYP2B6, CYP3A7 and CYP3A4 activities measured over time in 3D iHeps without (white bars) and after induction (black bars). Histograms represent mean±SD (n=3). D. CYP1A2 (EROD) and CYP3A4 (BROD) specific activities of 3D iHeps (white and black bars) and PHH spheroids (squared and chequered bars). Histograms represent mean±SD (n=8). E. UGTIAl activity of 3D iHeps (black bars) and PHH spheroids (white bars). Graph represents mean±SD (n=6). F. ADH activity of 3D iHeps (white and black bars) and PHH spheroids (squared and chequered bars). *** indicates p<0.001; ** indicates p<0.01; * indicates p<0.05.

FIG. 5. Functional characterization of iHeps.

Quantification of: A. Lactate detoxification and B. urea synthesis. Graphs on the left refer to quantification in 2D (white bars) and 3D (black bars) iHeps at day 28; graphs on the right refer to quantification in 3D iHeps (black bars) and PHH spheroids (white bars) over time. C. glycogenolysis in 2D (white bars) and 3D (black bars) iHeps at day 28. D. Scheme depicting the glucose metabolism analysis in 3D iHeps under hyperglycaemic and hypoglycaemic conditions. Graphs represent the glucose quantification after glycogenolysis (bottom left) and gluconeogenesis (bottom right) in 3D iHeps (black bars) and PHH spheroids (white bars). E. Phase 0-III metabolism in 2D and 3D iHeps. For the ICG uptake-release, test images were analysed with FIJI software. Briefly, images were converted into 32-bit grayscale then inverted. The mean grayscale analysis was performed using the analyse function. The results were normalized against the background and expressed as mean pixel values. Six independent experiments were performed for each condition and samples were analysed in triplicates. *** indicates p<0.001

FIG. 6. Bile acid secretion and bile canaliculi network analysis of 3D iHpes. A. Left panel: bile acid production and secretion by 3D iHeps in culture supernatants at day 20 (white bars) and 38 (black bars) of culture. Histograms represent mean ±SD (n=3). CA=cholic acid; DCA=deoxycholic acid; CDCA=chenodeoxycholic acid; GCA=glycocholic acid; GDCA=glycodeoxycholic acid; GCDCA=glycochenodeoxycholic acid; TCA=Taurocholic acid; TDCA=taurodeoxycholic Acid; TCDCA=taurochenodeoxycholic acid. Right panel: total bile acid production and secretion by 3D iHeps (black bars) and PHH spheroids (white bars) over time. Histograms represent mean±SD (n=3). B. Live imaging of the bile canaliculi (BC) network visualized through the excretion of the fluorescent probe DCFA in 3D iHeps at day 38. Scale bar=100 μm. C. Immunofluorescence staining of the apical membrane transporter BSEP in 3D iHep sections (75 μm) at day 38. Scale bar=75 μm. D. Left panel: 3D reconstruction of the BC network based on the apical membrane transporter BSEP staining in 3D iHep sections (75 μm) at day 38. Right panel: 3D reconstruction of a segment (ROI 1) of the BC network (rotation in x axis). E. Quantification and length measurements of bile canaliculi recorded on the surface (white bars) and in the core (black bars) of the 3D iHeps after DCFA treatment and BSEP staining.

TABLE 1
Summary table of the expression of hepatic markers recorded throughout the
maturation step, comparing the previously published protocol to the new
one. Results are expressed as quantification of the immunostaining positivity
of the samples. A direct comparison with the bibliographic data with human
foetal and adult hepatocytes is also given [36, 44, 47-49].

| Protein | Published protocol | | | New protocol 2D | | | New protocol 3D | | |
|---|---|---|---|---|---|---|---|---|---|
| expression | Day 22 | Day 25 | Day 28 | Day 22 | Day 25 | Day 28 | Day 22 | Day 25 | Day 28 |
| A₁AT | ++ | ++ | ++ | ++ | ++ | ++ | ++ | ++ | +++ |
| AFP | ++ | + | + | + | + | +/− | − | − | − |
| ALB | + | + | ++ | + | ++ | ++ | ++ | +++ | +++ |
| BSEP | − | + | + | + | ++ | ++ | ++ | +++ | +++ |
| CK19 | + | − | − | + | − | − | − | − | − |
| CK8 | − | + | + | + | + | ++ | ++ | ++ | +++ |
| CK7 | − | − | − | − | − | − | − | − | − |
| CLDN1 | + | + | + | + | + | ++ | ++ | ++ | ++ |
| CX26 | + | + | − | + | − | − | + | − | − |
| CX32 | − | + | + | + | + | ++ | ++ | ++ | +++ |
| CYP3A4 | − | + | + | + | ++ | ++ | + | ++ | +++ |
| ECADH | − | + | ++ | + | ++ | +++ | + | ++ | +++ |
| EPCAM | + | − | − | − | − | − | − | − | − |
| HNF1α | − | − | + | − | + | + | + | + | ++ |
| HNF4α | ++ | ++ | ++ | ++ | ++ | ++ | ++ | ++ | ++ |
| MDR1 | − | + | + | − | + | + | + | + | ++ |
| MDR3 | − | − | − | − | + | + | + | + | ++ |

TABLE 2
Summary of the functional features of the 2D and 3D iHeps generated with
the proposed protocol. A direct comparison with the bibliographic data
on human foetal and adult hepatocytes is also given [36, 44, 47-49].

| | Differentiated cells | | | |
|---|---|---|---|---|
| | 2D | 3D | Published data | |
| | Hepatocytes | Hepatocytes | Foetal | Adult |
| | (iHeps) | (iHeps) | Hepatocytes | Hepatocytes |
| AFP secretion | +/− | − | + | − |
| Albumin secretion | + | ++ | + | +++ |
| Glycogen synthesis | + | ++ | + | ++ |
| Glycogenolysis | + | ++ | + | ++ |
| (Glucose-6-Phosphatase activity) | | | | |
| Urea synthesis | + | ++ | + | ++ |
| Uptake/Secretion of bile | + | ++ | + | ++ |
| Phase 0-III metabolism: ICG uptake/Secretion | + | ++ | + | ++ |
| (expression of OATP1B3, NTCP and MRP-2) | | | | |
| CYP1A1/2 activity (EROD) | + | ++ | − | +++ |
| CYP3A7 activity | +/− | − | + | − |
| CYP3A4 activity (BROD) | + | ++ | − | +++ |

### EXAMPLE

### Material & Methods

### Differentiation Protocol

To induce hiPSC differentiation into iHBs, our already described protocol has been used with minor modifications [12,41]. Briefly, hiPSC colonies were enzymatically dissociated into single-cell suspension using 0.5% Trypsin-EDTA 1× solution (Gibco) then seeded at the density of 4.2×10⁴ cells/cm² on gelatine, at 37° C. and 5% CO2 in StemMACS™ iPS-Brew XF medium, supplemented with 10 mM Y-27632. When 70% of confluence was reached, the medium was replaced with RPMI-1640 medium (Gibco) complemented with B-27 serum-free supplement (Life technologies), 1× MEM non-essential amino acid solution (NEAA, Gibco) and 1× penicillin-streptomycin (Gibco) supplemented with 5 nM of CHIR99021 (Miltenyi Biotech) for 24 h. From day 1 to day 4, 100 ng/ml Activin A (Miltenyi Biotech) and 10 nM LY294002 (Sigma-Aldrich) were used to induce definitive endoderm. On day 5, 50 ng/ml Activin A (Miltenyi Biotech), 10 ng/ml bone morphogenetic protein 4 (BMP4) (R&D Systems) and 20 ng/mL FGF2 (Miltenyi Biotech) were added for three days to specify the hepatic endoderm. On day 9 and day 10, RPMI without methionine (Gibco) was supplemented with 20 ng/ml hepatocyte growth factor (HGF) (Peprotech) and 30 ng/ml fibroblast growth factor 4 (FGF4) (Peprotech). On day 11, the differentiated iHBs were detached enzymatically with StemPro Accutase Cell Dissociation Reagent (Gibco) and processed to induce their differentiation and maturation into iHeps.

Poly(dimethylsiloxane) (PDMS) moulds were purposely created as previously reported [42] and were constituted of 63 μ-cylinders of 1 mm diameter and depth. A 2% liquid agarose solution was poured into culture plates and the PDMS moulds were placed upside down and removed after cooling to create non-adherent uwells. To obtain spheroid formation 2.2×10⁵ iHBs were seeded per mould and incubated at 37° C. and 5% CO2 for 1 h. From this point onwards, Hepatocyte Complete Medium (HCM™) (Lonza) supplemented with 20 ng/ml HGF, 0.1 nM Dexamethasone (Dex) and 20 ng/ml Oncostatin M (OSM) was used and refreshed every other day for six days.

On day 18 of the differentiation protocol, in both 2D and 3D culture systems, alongside 20 ng/ml HGF, 0.1 nM Dex and 20 ng/ml OSM, 10 ng/ml of Vitamin K1 (Roche) was supplied and maintained until the end of the culture. From day 20, Dex was administered everyday so that its supply was decreased to 0.05 nM and reversed back to 0.1 nM every 24 h until the end of the culture. From day 22, 0.5 nM of Compound E (Santa Cruz Biotechnology) and 5 nM of SB431542 (Tocris Biosciences) were added to the medium while OSM concentration was decreased by half at every other day (10 ng/ml; 5 ng/ml; 2.5 ng/ml) until its complete removal.

The generated iHBs were also cultured in 2D and used as control for the differentiation protocol. Multiwell plates were coated with a homemade coating solution constituted of 1% w/v fibronectin (Sigma-Aldrich), 3% w/v calf skin collagen type I (Sigma-Aldrich) and 10%

w/v bovine serum albumin (BSA) (Sigma-Aldrich) [12]. Then, harvested iHBs were re-seeded at the density of 2×10⁵ cells/cm² and treated as for the 3D culture system.

Cryopreserved primary human hepatocytes (PHHs) (Biopredic international) have been also used as control to assess the maturation degree of the generated 3D iHeps. Cells have been thawed according to the provider's instructions, and processed as for self-assembling of iHBs. Spheroids were obtained after 48 h, the medium was refreshed every other day and PHH spheroids were maintained in culture for 8 days.

### Nucleic Acids Extraction and Gene Expression Assay

At established time point along the culture, the total RNA/DNA content of spheroids and 2D monolayers was extracted using TRIzol™ Reagent (Sigma-Aldrich Aldrich) and purified through the Direct-zol™ DNA/RNA MiniPrep Kit (Zymo Research) following the manufacturer's instructions. Quantification of RNA and DNA sample contents was performed by UV-visible Nanodrop Lite (ThermoFisher) and RT-PCR (reverse transcriptase-PCR) was performed using SuperScript™ First-Strand Synthesis System for RT-PCR (Invitrogen) with oligoDT primer and Platinium Taq DNA Polymerase (Invitrogen) following the manufacturer's instructions. For quantitative PCR analysis, the mRNA extraction and RT-qPCR conditions were set in line with the manufacturer's instructions. cDNAs were obtained using the SuperScript III kit (Invitrogen) with random hexamers. Three replicates per sample were analyzed for differential gene expression using the Mx3000P qPCR thermocycler system (Agilent) with brilliant III ultrafast SYBR Green (Agilent). Relative levels of expression were determined using the 2^{-ΔΔCt} method with GAPDH as the reference gene, and expression levels were described relative to foetal human hepatocytes (FHHs).

### In Vitro Assessment of Hepatocyte Function

For each test performed on hepatocyte function of the differentiated cells, DNA contents were used to quantify the cell number per sample so that quantification assays' results are shown for 10⁶ cells if not otherwise specified.

Albumin and α-Fetoprotein secretions. Samples of culture media were collected 24 h after every medium refreshing, centrifuged at high speed and supernatants were stored at −80° C. until measurements. AFP measurements were performed using AFP Human ELISA Kit (Fisher Scientific) following the manufacturer's instructions. Albumin secretion was quantified by the Human Albumin ELISA Quantification Set (Bethyl Laboratories) as instructed.

Urea production and Lactate metabolism assay. Fresh culture media supplemented with 1.5 mM NH4Cl and 2 mM L-Lactate were added to the samples and recollected after 3 hours. Urea secretion and lactate detoxification were assessed by QuantiChrome Urea Assay Kit (BioAssay Systems) and Lactate Assay Kit (Sigma) respectively according to the manufacturer's instructions.

Cytochrome P450 activity (Phase I metabolism). To measure cytochrome P450 activity, specifically the isoforms 1A1, 1A2, 3A7, 3A4, and 2B6. the P450-Glo™ Assay (Promega) was used following the manufacturer's instructions. Briefly, samples from 2D and 3D cultures were incubated with luminogenic (luciferin) cytochrome P450 substrates. The amount of light produced is directly proportional to cytochrome P450 activity. Furthermore, to directly quantify the CYP1A1/2 and CYP3A4 isoform activities responsible for Phase I metabolism of xenobiotics, cells cultured in both monolayer or spheroids were treated with or without 10 μM rifampicin (RMP) or omeprazole (OMP) (Sigma-Aldrich) for 48 hours to assess the inducibility of the cytochromes. 8 μM 5-ethoxyresorufin and 8 μM 7-benzyloxyresorufin were used, respectively, as substrates for the two isoforms. To inhibit phase II CYP450 enzymes, a 3 mM salicylamide (Sigma-Aldrich) and 10 μM dicumarol (Sigma-Aldrich) treatment was carried out on each sample. Supernatants were collected and the metabolite resorufin was quantified using a fluorescence microplate reader at 595 nm (Spectafluor Plus, TECAN).

Uridine diphospho-glucuronosyl transferase 1A1 activity (Phase II metabolism). The UGT1A1 activity was assessed by quantifying the glucuronidation of 4-Methylumbelliferone (4-MU) (Sigma-Aldrich). 3D culture samples (iHeps and PHHs) were treated with 100 μM 4-MU for 24 h then the supernatants were collected and the metabolite was quantified using a fluorescence microplate reader at 450 nm (Spectafluor Plus, TECAN).

Alcohol detoxification (Phase II metabolism). To evaluate alcohol metabolism in 3D iHeps and PHH spheroids, the Alcohol Dehydrogenase (ADH) Activity Colorimetric Assay Kit (CliniSciences) was used in accordance with the manufacturer's instructions. 3D samples were tested with and without 10 mM EtOH.

Glycogen Glucose metabolism. iHeps in 2D and 3D culture were investigated to assess the glycogen storage and glucose homeostasis regulation. Briefly, at specific time points along the 2D and 3D cultures, cells were treated for 3 h with a homemade low-glucose medium with or without 10 nmol/L glucagon (Sigma-Aldrich) to induce glucose release from the glycogen storage. Media samples were then collected and glucose concentration was determined using the High Sensitivity Glucose Assay Kit (Sigma). Periodic acid-Schiff (PAS) staining was then carried out on 2D and 8 μm cryosections of the very same samples. Specimens were fixed for 10 minutes at room temperature with 4% formol then treated with 1% periodic acid solution (Sigma-Aldrich) for 10 minutes. After washing, slides were immersed in Schiff's reagent (Fisher Scientific) for 30 minutes at RT. Before counterstaining with Mayer's Hematoxylin (Sigma), samples were washed carefully with Scott's tap water.

To investigate further, 3D specimens (iHeps and PHHs) were incubated for 4 h with 25 mM D-Glucose and 100 nM insulin to mimic hyperglycemia. Glycogen synthesis and storage were then determined using the Periodic Acid-Schiff (PAS) Staining Kit (Sigma-Aldrich) as described above. The following day, about 500 spheroids were incubated in a glucose-free medium supplemented with 10 nmol/L glucagon (Sigma-Aldrich) in order to mimic short fasting conditions and assess glycogenolysis. Furthermore, to evaluate the capacity for gluconeogenesis, a long fasting state was mimicked by culturing samples without glucose but supplemented with 2 mM of pyruvate for 15 h. The media were then collected, and glucose levels determined using the High Sensitivity Glucose Assay Kit (Sigma).

Indocyanine green (ICG) uptake and excretion. To monitor Indocyanine green (ICG) uptake and excretion in the spheroids and the 2D cultures, Cardiogreen (Sigma-Aldrich) was dissolved in DMSO to a concentration of 32 mM and then diluted in HBM™ to 5 μM. Samples were incubated for 30 minutes in ICG/HCM solution at 37° C. and ICG excess was removed by carefully washing. Images of cultured cells and spheroids were taken under an inversed light microscope after 15-, 30- and 60-minutes post washing and at the final time points of 3 and 6 hours. The images were analysed by Fiji software. Pictures were first converted into 16-bit grayscale then inverted and the mean grayscale of cells and spheroids was performed using the analyse function. The results were expressed as mean pixel values.

Bile acid production and transport (excretion). The production of bile acids (BAs) by spheroids (iHeps and PHHs) were investigated by liquid chromatography-tandem mass spectrometry (LCMS/MS) on cell lysates and culture supernatants. Moreover, the total BA content was quantified in both 3D iHeps and PHH spheroids using the Total Bile Acid (TBA) (Human) ELISA Kit (BioVision) in accordance to the manufacturer's instructions.

Bile canaliculi assessment and 3D imaging. To investigate and assess the formation and development of bile canaliculi structures in both 2D and 3D cultures, samples were treated with DCFA probe (Abcam). Briefly, samples were incubated with 5 μM probe solution for 30 minutes and after washing, images of cultured cells and spheroids were taken under the microscope in the GFP channel. The resulting image dataset and the immunofluorescence images obtained for the bile salt export pump (BSEP) staining were then processed using Fiji software to obtain segmentation and accurate 3D reconstructions of the bile canaliculi network. Specifically, images were pre-processed for photo-bleaching and noise correction using Bleach correction and Gaussian smooth (Fiji), and then Volume viewer and 3D viewer (Fiji) were used to complete the imaging. A geometrical analysis was furthermore performed on the skeleton of the bile network and the average lengths and the branching topography of the canaliculi in each sample were quantified. Five samples for 2D and 3D cultures were analysed for a total of six independent experiments and, to clearly evaluate if the bile canaliculi extend like a network-like structure into the core of the spheroids, some samples were cryopreserved and 5 μm sections were stained specifically with anti-BSEP and anti-Albumin.

### Statistics

Results were expressed in mean +/−standard deviation. Statistical analysis was performed by one-way ANOVA with Newman-Keuls test for multiple comparisons. *** indicates p<0.001; ** indicates p<0.01; * indicates p<0.05

### Results

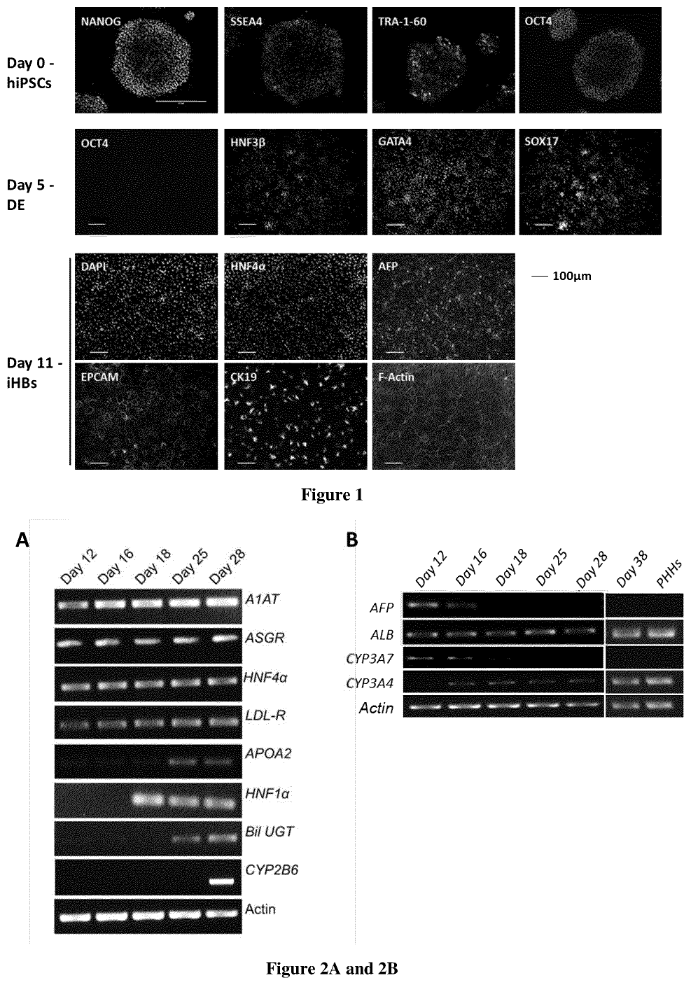

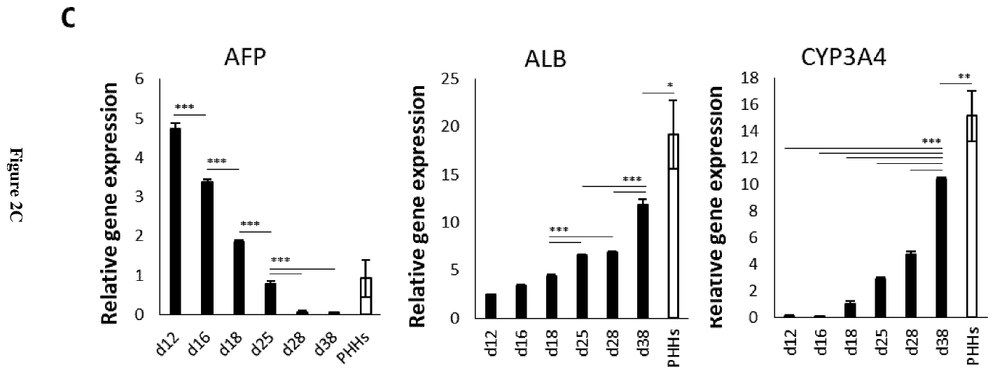

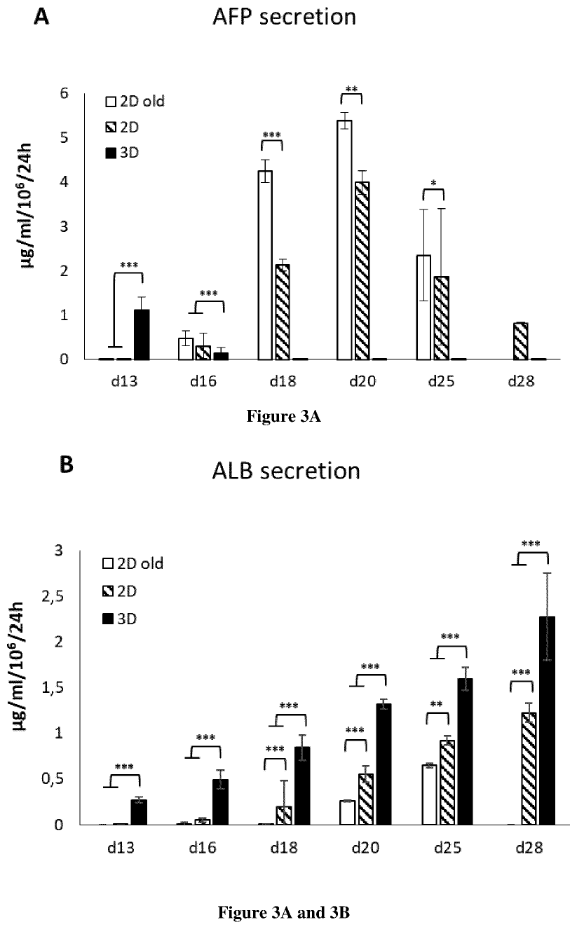

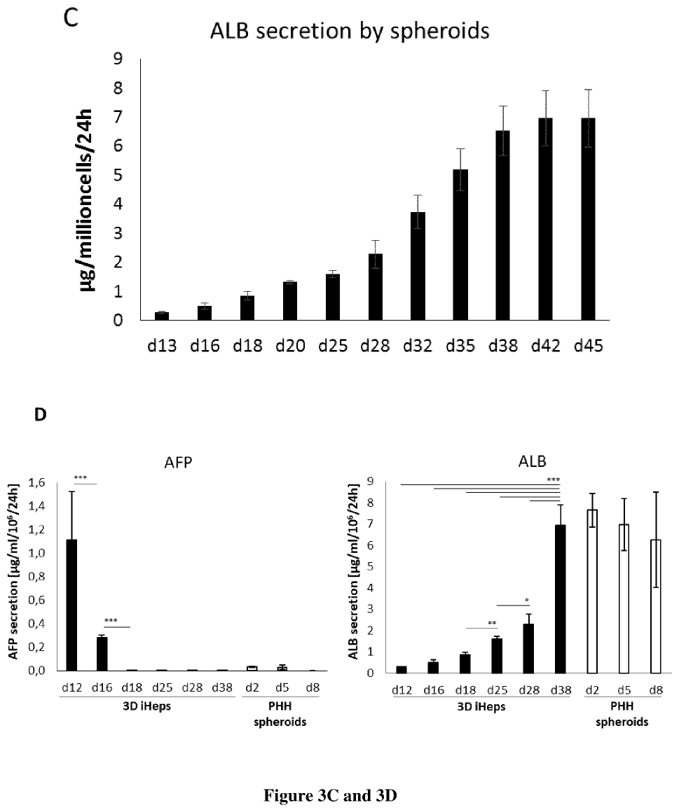

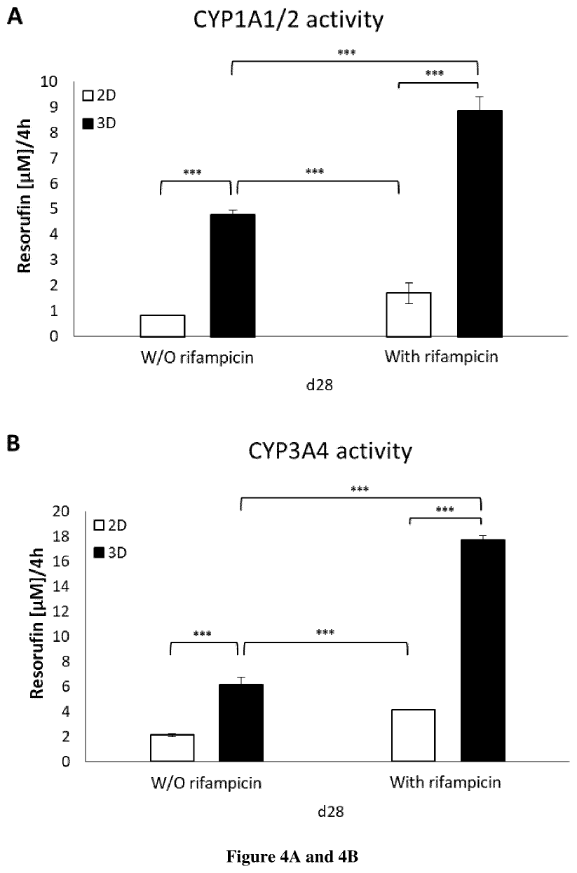

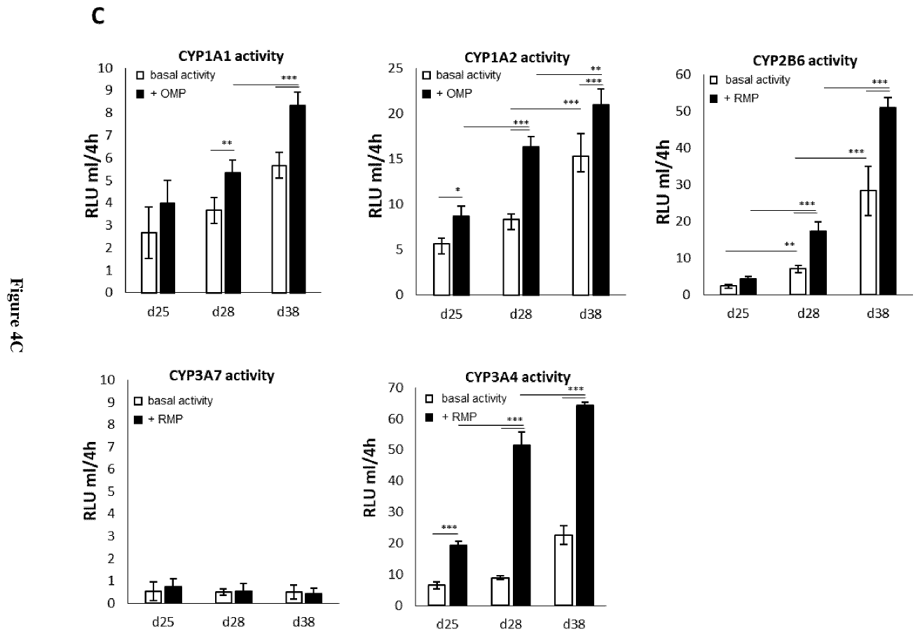

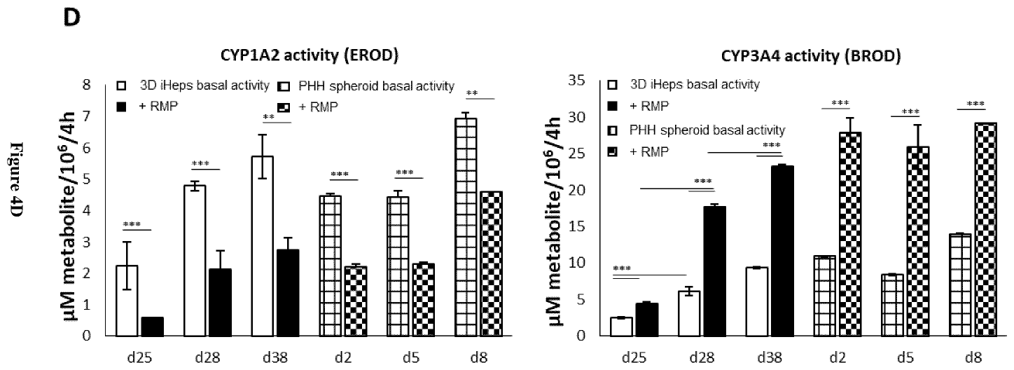

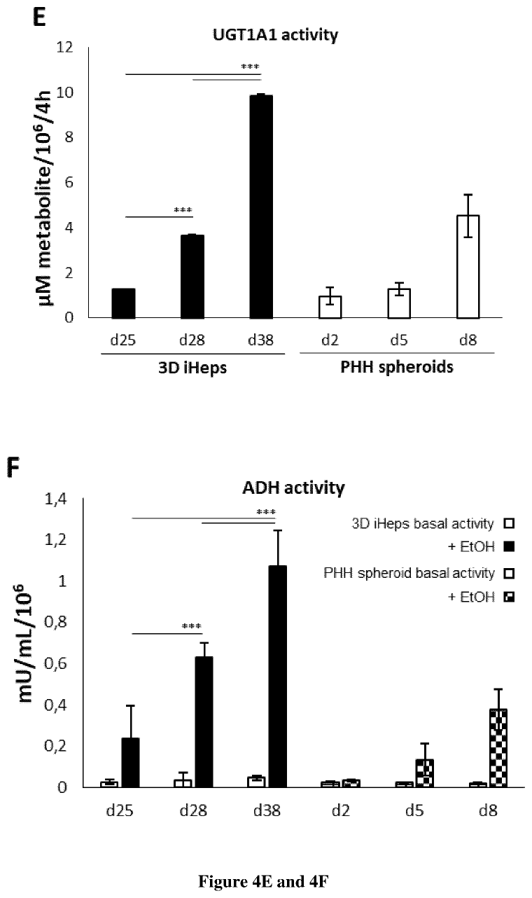

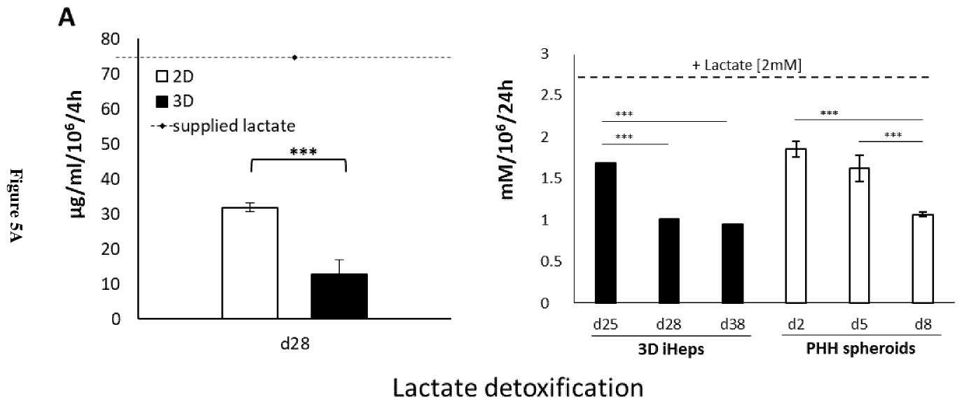

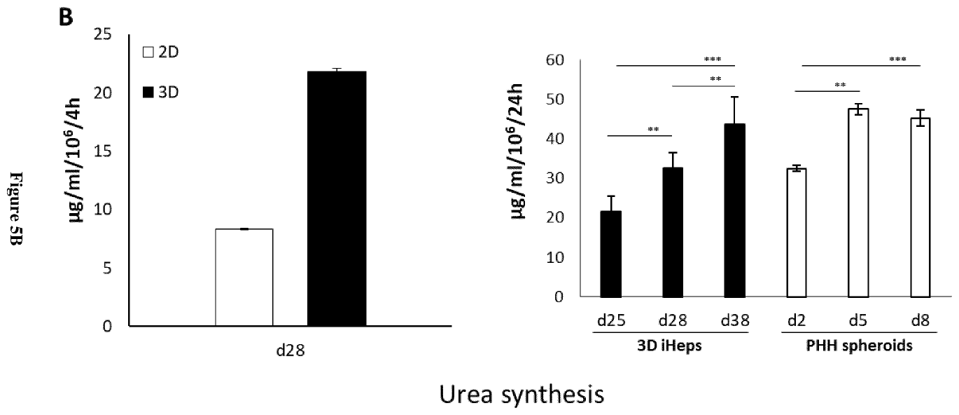

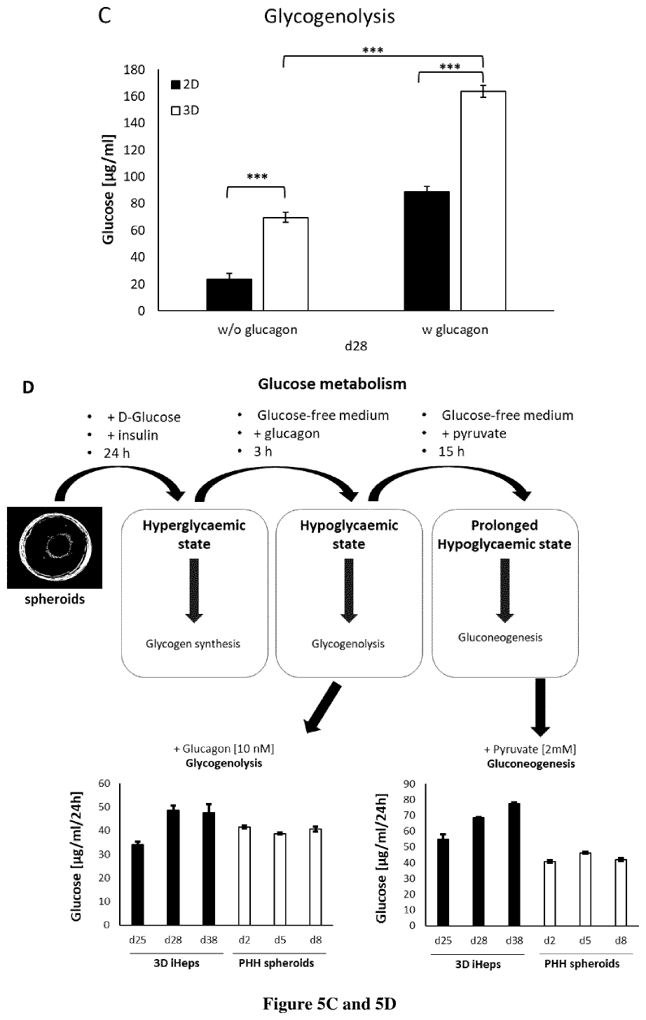

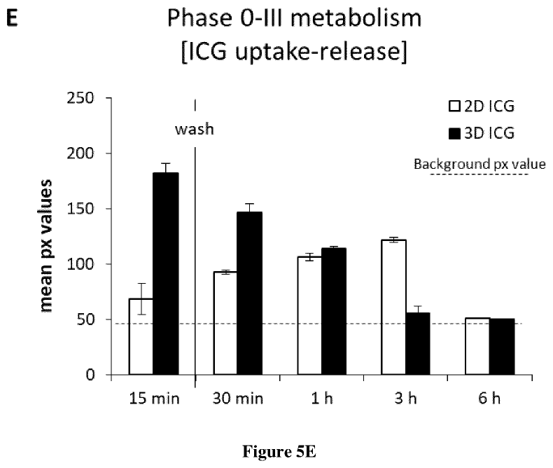

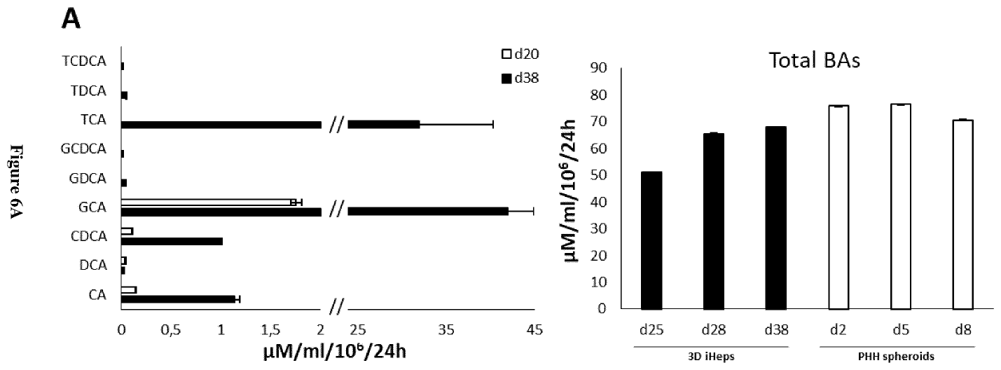

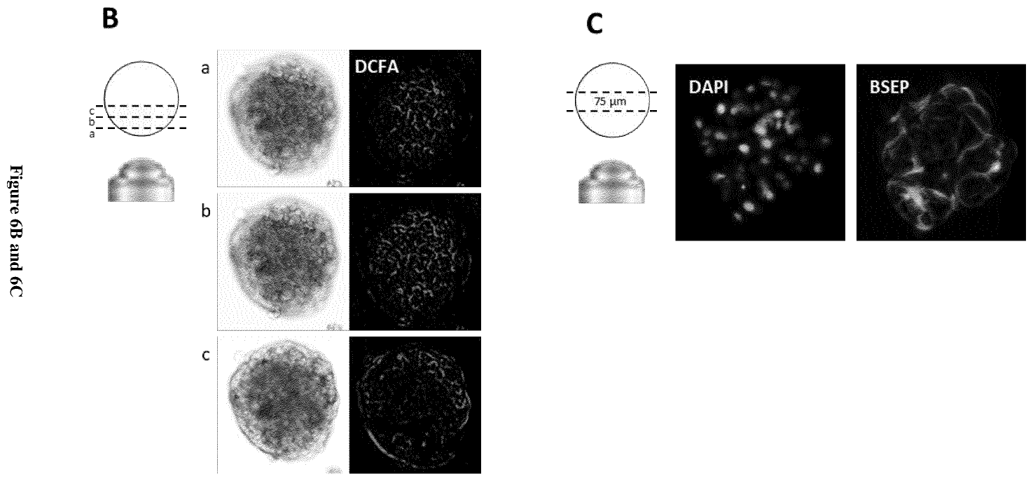

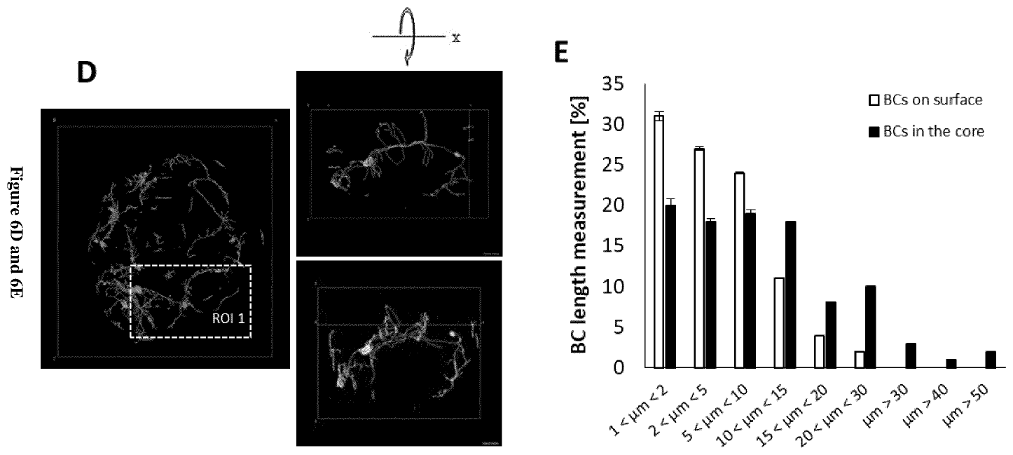

The differentiation protocol consists of three major steps. Human iPSCs were progressively differentiated in definitive endoderm cells, hepatic progenitors, the hepatoblasts (iHBs), and hepatocytes (iHeps). The morphological analysis of the cells obtained in 2D showed that within eleven days of treatment, hiPSCs progressively differentiated in well-defined iHBs. iHBs then further evolved and acquired, by day 18 of differentiation, the typical polygonal shape of hepatocytes, with bright and well-defined membranes and the occasional presence of bi-nucleated cells. Immunofluorescence analysis confirmed the efficiency of the differentiation protocol. Stemness and pluripotency markers NANOG, SSEA4, TRA-1-60, and OCT4 expressed in hiPSCs progressively disappeared and, at day 11, cells were positive for HNF4α, AFP, EPCAM and, cytokeratin 19 (CK19) as evidence of their differentiation into iHBs (FIG. 1). iHeps were maintained for a total of twenty-eight days in culture. Furthermore, 3D configuration allowed extended iHep culture until day 45 (34 days of 3D culture). Gene expression profile analysed using RT-PCR (FIG. 2A) revealed the expression of hepatic markers as soon as 24 h of 3D culture (day 12); these included mRNAs for α1-antitrypsin (A1AT), asialoglycoprotein receptor (ASGR), HNF4α, low density lipoprotein receptor (LDL-R), and apolipoprotein AII (APOA2). mRNAs for HNF1α, bilirubin UDP-glucuronosyltransferase (Bil-UGT), and CYP2B6 were also expressed from days 15, 18, 22, and 28, respectively, thus confirming 3D iHeps maturation over time. Most remarkably, AFP mRNA expression in iHep-Orgs vanished completely within one week of culture (day 18), while ALB mRNA was already expressed within 24 h. Likewise, a decrease in CYP3A7 mRNA levels and the growing expression of CYP3A4 were recorded (FIG. 2B). Quantitative PCR (qPCR) confirmed these findings (FIG. 2C) with a loss of AFP mRNA within two weeks of 3D culture, whereas the expression of albumin and CYP3A4 increased over time, reaching at day 38 65% and 68% of PHH level respectively. Enzyme-linked immunosorbent assay (ELISA) performed on cell supernatants confirmed the previously observed profiles recorded by RT-PCR concerning AFP and albumin kinetics of expression in 3D cultures (FIG. 3A-3C). The complete disappearance of the AFP secretion was detected within the first week of 3D culture (day 18), confirming that iHeps acquired an adult mature phenotype. However, in 2D samples, the AFP profile showed the typical bell curve already observed in literature in which the highest secretion point was recorded around day 20 of differentiation. Moreover, direct comparison with the results obtained from our previously published protocol showed that, although the AFP secretion kinetics still show a bell-shaped profile with a peak recorded on day 20, the 2D iHeps generated through the new protocol also statistically decreased AFP secretion (FIG. 3A). The albumin secretion recorded in spheroid cultures was 3-fold higher with respect to the 2D system already at day 16 of differentiation. Indeed, the protein secretion in 3D was detected from day 13 of culture and reached a value of 3 μg/millioncells after 28 days of culture versus 1.3 μg/millioncells secreted by 2D iHeps. Moreover, the higher albumin secretion recorded in 2D cultures was statistically significant when compared to data obtained with the already published protocol (FIG. 3B). Indeed, albumin was detected from day 16, with increasing values along the time. On the contrary, 2D iHeps obtained through the previously published protocol were able to secrete the protein from day 20 of culture. Taken together, AFP and albumin data secretions are evidence of a quicker and marked improvement of the differentiation and maturation process of the iHeps both in 2D and in the 3D culture systems with respect to the data previously reported by us and others in the literature. The development of the 3D culture system allowed prolonging the differentiation until, at least, day 45, resulting in a prolonged plateau of albumin secretion reaching 7 μg/millioncells/24 h (FIG. 3C). Moreover, the average values for AFP and ALB secretions documented for 3D iHeps at day 38 were comparable to that recorded for PHH spheroids (FIG. 3D). To qualitatively characterize the iHeps obtained with our new protocol at day 28, immunofluorescence analysis was carried out on 3D cryo-preserved slices (7 μm) and full 3D spheroids (data not shown). Hepatic markers such as HNF4α, ALB, cytokeratin 8 (CK8), alpha 1-antitrypsine (A1AT), CYP3A4, asialoglycoprotein receptor (ASGR), claudin-1 (CLDN1), HNF1α, E-Cadherin (ECADH), connexin 32 (CX32), Zonula occludens-1 (ZO1), and the Bile Salt Export Pump (BSEP) were detected. Furthermore, 3D differentiated cells displayed a mature profile, as shown by the absence of AFP and the homogeneous staining of all hepatic markers including MDR-1 and -3, UGT1A1 and BSEP which stated that iHeps acquired the typical hepatocyte polarization (basolateral/apical). In order to achieve a broad characterization of the functional maturation of our iHeps, we evaluated the cytochrome P450 activity of the spheroids. The iHeps in spheroids showed a 3-fold higher metabolic activity compared to the iHeps obtained in 2D culture systems. The specific activity of the isoform CYP1A1/2 and CYP3A4 were examined to evaluate the xenobiotic phase I metabolism of our iHeps (FIG. 4A and 4B). As expected, activity recorded for 3D samples was 3-6 times higher with respect to the controls at day 28 of culture. The cytochrome isoforms 1A1/2 and 3A4 were able to metabolise, in four hours, around 55% of the supplied drugs, and after rifampicin induction, the total activity increased by 30%. All data recorded for spheroids were statistically significant compared to the 2D system. To further deepen our analysis, the enzymatic activities of CYP1A1, 1A2, 2B6, 3A7, and 3A4 were examined at different time of the culture to evaluate xenobiotic phase I metabolism. As expected, spheroids already displayed detoxification capabilities by day 25, and their performance improved over time. Only CYP2B6 activity appeared later on, thus confirming the data obtained from gene expression analysis. Moreover, all the isoforms investigated responded to the induction treatment (FIG. 4C). Significantly, the activity of CYP3A7, the foetal isoform of CYP3A4, was the only enzyme whose recorded activity values were close to zero, according to the RT-PCR results. This again indicates that the spheroids had reached a significant level of maturation (FIG. 4C). To compare 3D iHeps with PHH spheroids, CYP1A2, and CYP3A4 activities were also specifically examined using EROD and BROD specific tests. Both isoforms in iHep spheroids were able to metabolize the drug supplied and responded to rifampicin treatment by a 50% reduction and 60% increase in CYP1A2 and CYP3A4 detoxification capabilities, respectively (FIG. 4D). Further confirmation of the high level of functional maturation achieved by 3D iHeps, their metabolic capacities closely mirror those of PHH spheroids. Glucuronidation through UGT1A1 (phase II metabolism) and the detoxification of ethanol by alcohol dehydrogenase (ADH) are specific features of adult hepatocytes. 3D iHeps treated with 4-MU and EtOH displayed both UGT1A1 and ADH activity as early as day 25 of culture, and a significant increase in detoxification capabilities over time. At day 38 of differentiation, a 3-fold higher concentration of conjugated metabolite was detected (FIG. 4E) and importantly, the spheroids already displayed increased ADH activity by day 25 of culture (FIG. 4F), thus proving them to be capable of metabolizing ethanol in an inducible manner. Unexpectedly, as early as day 25, iHep spheroids proved to be functionally comparable or superior to PHH spheroids for both enzymatic activities.

Some of the important liver-specific functions, such as lactate metabolism, urea synthesis, glycogenolysis, and phase 0-III metabolism, were investigated. When evaluating the lactate detoxification (FIG. 5A) and urea synthesis upon ammonium challenge (FIG. 5B), iHeps in spheroids displayed 30% higher detoxification capacity and three-fold urea production ability than iHeps differentiated in 2D. Again, data recorded for spheroids were comparable or superior to PHH spheroids. Moreover, iHeps differentiated in 3D were responsive to the hormonal stimulus given by glucagon, depleting their glycogen storage points (FIG. 5C). When subjected to glucose deprivation, spheroids released free glucose in the medium. About 200 μg/ml of sugar were quantified in 3 h after hormone treatment, which corresponds to a 3- and 4-times higher glycogenolysis process with respect to the basal release (w/o glucagon) and the 2D controls respectively (FIG. 5C). More deeply we investigated the glycogenolysis and gluconeogenesis capabilities of the iHep spheroids as they mimicked in vitro both a short-lived and lengthy fasting state. Under hyperglycaemic conditions and insulin stimulation, iHep spheroids were able to respond through glucose anabolism, as assessed by the glycogen storage observed (data not shown). Moreover, in the absence of glucose (hypoglycaemic state) and in the presence of glucagon, the organoids were also able to catabolize the glycogen accumulated over the previous 24 h and then release it as glucose into the medium (FIG. 5D). Likewise, spheroids responded to a prolonged hypoglycaemic state. Using pyruvate as the only source available, they activated gluconeogenesis and released glucose into the medium. As for UGT1A1 and ADH activities, 3D iHeps also proved to be functionally comparable or superior to PHH spheroids in both glycogenolysis and gluconeogenesis performances. The indocyanine green (ICG) uptake/release test, carried out on living samples, showed that iHeps expressed the OATP1B3 (organic anion-transporting polypeptide 1B3) and NTCP (Na+-taurocholate co-transporting polypeptide) transporters, responsible for the clearance of bile acids and organic anions from the liver, on their basolateral membranes (FIG. 5E). 2D and 3D samples were able to intake ICG within 15 and 30 minutes respectively. After washing spheroids showed a 4-fold quicker release of the dye compared to the 2D culture system. In details, monolayer staining persisted five hours after washing whereas 3D samples were able to excrete the molecule already within 30 minutes from the dye administration, as evidence of a quicker metabolizing activity and a significantly higher expression of the apical transporter MRP-2 (Multidrug resistance-associated protein 2) in charge of the hepatobiliary excretion of the organic anions and conjugated organic anions. Bile acids (BA) production and secretion were detected in spheroids already by day 20 (data not shown) in both cell lysates and culture supernatants. Cholic acid (CA) and chenodeoxycholic acid (CDCA) were indeed secreted at three weeks of culture, alongside glycocholic acid (GCA). By day 38, a significant increase was observed in the secretion of CA and CDCA, the major primary bile acids synthesized in the human liver, and their conjugated forms with taurine (TCA and TCDCA) or glycine (GCA and GCDCA) (FIG. 6A, left panel). These findings thus attested to the correct expression and activity of both microsomal enzyme cholesterol 7α-hydroxylase (CYP7A1) and bile acid coenzyme A: amino acid N-acyltransferase (BAAT). Moreover, in terms of total BA secretion, no significant differences were recorded between the metabolic capacities of iHep spheroids and PHH spheroids on day 28 (FIG. 6A, right panel). Using the fluorescent probe DCFA, which is a tracer of MRP2 transport, we also observed the development of a network of elongated bile canaliculi (BCs) at the surface of the 3D iHeps (FIG. 6B). In view of these results, we wondered if the bile canaliculi (BC) network might extend to the core of the organoids. We therefore labelled 75-μm-thick slices of the DCFA-treated iHep spheroids for BSEP expression and analysed the datasets obtained after immunostaining. The Z-stack reconstruction did indeed confirm our hypothesis. As shown in FIG. 6C, BCs looked as well-defined and finely organized structures that appeared to involve a fair number of cells. Images and representative regions of these slices were also further processed to obtain 3D reconstructions of the network thus highlighted. This in-depth investigation clearly showed that a three-dimensional and complex system of branching canaliculi ex-tended within the organoids (as shown in FIG. 6D). Measurements of the BC revealed that this network was mostly made up of 2 μm-, 5 μm-, and 10 μm-long canaliculi (82%) on the iHep spheroids surface and only 6% of the structures far exceeded these lengths. By contrast, in the cores, the percentage of small branches shifted significantly (57%) towards longer structures. In particular, 6% of the BC detected were longer than 40 μm (FIG. 6E).

### Discussion

The present study describes the improvement of our already published differentiation protocol for the generation of iHeps with mature features. By self-assembling hepatic progenitors into spheroids and by refining the maturation step of our differentiation protocol, we aimed at generating iHeps with an improved maturity degree, showing morphological and functional features of adult hepatocytes. We first proceeded with the regular administration of Vitamin K1, the major dietary source and primary circulating form of Vitamin K, during our maturation protocol, alongside the fine regulation of the glucocorticoid supplement. Moreover, we decided to slowly remove the OSM, a member of the IL-6 family secreted from hematopoietic cells proliferating in the foetal liver. Indeed, OSM strongly enhanced differentiation of foetal hepatocytes [13] but its concentration declines with the migration of hematopoietic cells to the bone marrow during the organogenesis allowing the hepatocyte maturation.

Human iPSCs were differentiated into HBs within ten days and showed all the characteristics of the hepatic lineage. Indeed, cells were positive for HNF3β, HNF4α, AFP and CK19 staining. Moreover, iHeps generated in 2D, showed the acquisition of a polygonal morphology as expected from adult hepatocytes. Using non-adherent uwells, hepatic progenitors were able to re-arrange in a 3D environment and self-assemble as spheroids, which were selectively guided to differentiate into iHeps. The expression, or lack of expression, of some genes and proteins throughout the differentiation process have been investigated and the overall analysis confirmed the correct engagement of the differentiation towards hepatocytes.

Albumin secretion and the disappearance of AFP production are some of the hallmarks to discriminate foetal and adult hepatocytes. The complete disappearance of AFP mRNA expression in spheroids prompted us to thoroughly investigate the secretion of these two proteins over time. Our data showed that AFP secretion completely disappeared within six days of 3D culture while albumin secretion regularly increased over time, confirming an improved maturation of the hepatocytes. About 2.5 μg/10⁶/24 h of albumin were quantified after two weeks of 3D culture, and more importantly, spheroids were able to maintain hepatic features and secretion functions for over two more weeks. In contrast with the classic 2D systems, in which differentiating iHeps can be susceptible to detachment within three weeks from the seeding, 3D samples allowed to extend the cultures for a total of 45 days. 3D iHeps were able to secrete between 6 and 8 μg/10⁶/24 h of albumin. One major issue in culturing iHeps is certainly related to the stability of their phenotype in long-term culture [38]. Values recorded are in line with the albumin expressed by most primary human hepatocytes (PHHs) in both 2D and 3D culture systems [44,47,48]. The robustness of the albumin secretion data suggests that the iHeps may have a major advantage over PHHs, which are known to rapidly lose functions in conventional 2D cultures [33]. Indeed, to improve the hepatocyte-specific functions in culture, approaches that foresee cell-matrix interactions and co-culture systems [31] are currently used. Our culture system relies only on the ability of the cells to spontaneously aggregate and then self-organize in a functionally mature structure, without any external intervention such as matrices or other cell types. By optimizing the growth factor supplies (the vitamin K1, the modulation of dexamethasone concentrations and the removal of OSM) in combination with our 3D culture system, we improved the differentiation of iHeps which over time proved to be very similar in function to PHH spheroids.

Another important marker of the improved maturation of iHeps is the extinguished expression of the foetal enzyme CYP3A7 mRNA with the concurrent appearance of CYP3A4 mRNA expression. Moreover, immunofluorescence staining of full and sliced spheroids showed a homogeneous expression of hepatic markers such as HNF1α, CK8, ASGR, A1AT, and ECADH as well as MDR1 and 3, BSEP, ZO1, CLDN1, and CX32, whose patterns suggest their specific distribution on the apical or basolateral membranes of the cells [50]. Connexin (CX) expression patterns undergo stage-dependent changes during hepatic differentiation. Therefore, it is an important hallmark to determine the hepatocyte maturation stage along the time. Hepatic progenitor cells switch from CX43 to CX26 expression and, to CX32 expression upon differentiation into hepatocytes, both in vivo and in vitro. CX32 establishes, in the adult liver, an elaborated network between hepatocytes and its expression is essential for most of the hepatic functions as glycogenolysis, albumin secretion, ammonia detoxification, CYP-mediated xenobiotic biotransformation, and bile secretion [48]. The latter depends on the correct acquisition of membrane polarity. Bile and waste products are excreted through bile canaliculi characterized by tight junction proteins that seal off the bile from the cells.

Alongside the assessment that 3D iHeps are able of bile acid production and secretion rate similar to PHH spheroids, the BSEP expression highlighted through immunofluorescence analysis in the core of our 3D iHep suggested the possible development of a bile canalicular network. Then, a computational (spatial) reconstruction was carried out using DCFA analysis. DCFA is widely used to assess the production/secretion process of toxic metabolites through the bile canaliculi system. Spheroids showed a considerable ability to intake DCFA and also to metabolize and secrete it correctly within 3 hours. It is well known that the hepatic transport system is not fully functional at the foetal stage because the proteins responsible for the bile transport, secretion and excretion are not or not entirely expressed. Thus, recorded data, suggest that at least functional characteristics of post-natal hepatocytes have been correctly acquired by the iHeps [50]. Although some published protocols have confirmed the expression of apical markers in human iHeps, to our knowledge, no study as yet had assessed development of the BC network into the core of hepatic organoids.

Other functionalities required by mature iHeps are urea secretion, lactate detoxification, lipid and glucose storages, and drug-metabolizing activity, including the phase 0 and III metabolisms carried out by specific transporters such as MRP2, OATPs and NTCPs.

3D iHeps were shown to be able to metabolize pathological concentration of lactate and ammonia, markedly lowering their levels in the culture medium as expected from mature highly functional hepatocytes. Noteworthy was the ability of iHeps to respond to hormone-induced hyperglycaemic and hypoglycaemic conditions. The glycogen storage points, detected in the spheroids by PAS staining (Periodic acid-Schiff Stain) (data not shown), were promptly degraded when a low glucose medium was supplied, confirming the expression and the functionality of the GLUT2 channels in the iHep membranes, which allow glucose to exit the cell via facilitated diffusion, as well as the expression of the two most important enzymes involved in the process, the glycogen phosphorylase and the glucose-6-phosphatase. The latest is specific for hepatic cells since it is not present in myocytes, where glycogenolysis also takes place.

Analysis of the ICG uptake/release process confirmed the expression of both apical and basal transporters as MRP2 (ABCC2), OATPs and NTCPs, whose expression is dependent on the transcription factor HNF1α [38,50].

Taken together, these results indicate that iHeps reached an increased maturation degree compared to previously reported data (Table 1 and 2). The removal of OSM along with the regular supply of Vitamin K1 and the corticosteroid intake control resulted in the acquisition of functions that surpass foetal hepatocytes (Table 2) and other reported iPSC-derived HLCs [47]. The proper maturation of iHeps and their phenotypic stability are of great importance for iHep applications like disease modelling, drug screening, toxicology studies, liver bioengineering or BAL development. The iHeps generated with our protocol represent a homogenous cell population with high similarity to adult hepatocytes.

### REFERENCES

Throughout this application, various references describe the state of the art to which this invention pertains. The disclosures of these references are hereby incorporated by reference into the present disclosure.
[1] Angelos M G, Kaufman D S. Pluripotent stem cell applications for regenerative medicine: Current Opinion in Organ Transplantation 2015:1. https://doi.org/10.1097/MOT.0000000000000244.
[2] Yi F, Liu G-H, Belmonte J C I. Human induced pluripotent stem cells derived hepatocytes: rising promise for disease modeling, drug development and cell therapy. Protein Cell 2012;3:246-50. https://doi.org/10.1007/s13238-012-2918-4.
[3] Hannoun Z, Steichen C, Dianat N, Weber A, Dubart-Kupperschmitt A. The potential of induced pluripotent stem cell derived hepatocytes. Journal of Hepatology 2016;65:182-99. https://doi.org/10.1016/j.jhep.2016.02.025.
[4] Messina A, Luce E, Hussein M, Dubart-Kupperschmitt A. Pluripotent-Stem-Cell-Derived Hepatic Cells: Hepatocytes and Organoids for Liver Therapy and Regeneration. Cells 2020;9:420. https://doi.org/10.3390/cells9020420.
[5] Chen S, Wang J, Ren H, Liu Y, Xiang C, Li C, et al. Hepatic spheroids derived from human induced pluripotent stem cells in bio-artificial liver rescue porcine acute liver failure. Cell Res 2020;30:95-7. https://doi.org/10.1038/s41422-019-0261-5.
[6] Boyd A, Newsome P, Lu W-Y. The role of stem cells in liver injury and repair. Expert Review of Gastroenterology & Hepatology 2019; 13:623-31. https://doi.org/10.1080/17474124.2019.1618186.
[7] Li Y, Meng Q, Yang M, Liu D, Hou X, Tang L, et al. Current trends in drug metabolism and pharmacokinetics. Acta Pharmaceutica Sinica B 2019;9:1113-44. https://doi.org/10.1016/j.apsb.2019.10.001.
[8] Holmgren G, Ulfenborg B, Asplund A, Toet K, Andersson C X, Hammarstedt A, et al. Characterization of Human Induced Pluripotent Stem Cell-Derived Hepatocytes with Mature Features and Potential for Modeling Metabolic Diseases. IJMS 2020;21:469. https://doi.org/10.3390/ijms21020469.
[9] Trefts E, Gannon M, Wasserman D H. The liver. Current Biology 2017;27:R1147-51. https://doi.org/10.1016/j.cub.2017.09.019.
[10] Touboul T, Chen S, To C C, Mora-Castilla S, Sabatini K, Tukey R H, et al. Stage-specific regulation of the WNT/β-catenin pathway enhances differentiation of hESCs into hepatocytes. Journal of Hepatology 2016;64:1315-26. https://doi.org/10.1016/j.jhep.2016.02.028.
[11] Lv L, Han Q, Chu Y, Zhang M, Sun L, Wei W, et al. Self-renewal of hepatoblasts under chemically defined conditions by iterative growth factor and chemical screening. Hepatology 2015;61:337-47. https://doi.org/10.1002/hep.27421.
[12] Caron J, Pène V, Tolosa L, Villaret M, Luce E, Fourrier A, et al. Low-density lipoprotein receptor-deficient hepatocytes differentiated from induced pluripotent stem cells allow familial hypercholesterolemia modeling, CRISPR/Cas-mediated genetic correction, and productive hepatitis C virus infection. Stem Cell Res Ther 2019;10:221. https://doi.org/10.1186/s13287-019-1342-6.
[13] Touboul T, Hannan N R F, Corbineau S, Martinez A, Martinet C, Branchereau S, et al. Generation of functional hepatocytes from human embryonic stem cells under chemically defined conditions that recapitulate liver development. Hepatology 2010;51:1754-65. https://doi.org/10.1002/hep.23506.
[14] Tolosa L, Caron J, Hannoun Z, Antoni M, López S, Burks D, et al. Transplantation of hESC-derived hepatocytes protects mice from liver injury. Stem Cell Res Ther 2015;6:246. https://doi.org/10.1186/s13287-015-0227-6.
[15] Fourrier A, Delbos F, Menoret S, Collet C, Thi Thuy L T, Myara A, et al. Regenerative cell therapy for the treatment of hyperbilirubinemic Gunn rats with fresh and frozen human induced pluripotent stem cells-derived hepatic stem cells. Xenotransplantation 2019:e12544. https://doi.org/10.1111/xen. 12544.
[16] Kamiya A, Kinoshita T, Miyajima A. Oncostatin M and hepatocyte growth factor induce hepatic maturation via distinct signaling pathways. FEBS Letters 2001;492:90-4. https://doi.org/10.1016/S0014-5793(01)02140-8.
[17] Kamiya A, Gonzalez F J. TNF-? regulates mouse fetal hepatic maturation induced by oncostatin M and extracellular matrices. Hepatology 2004;40:527-36. https://doi.org/10.1002/hep.20362.
[18] Kido T, Koui Y. Induction of Functional Hepatocytes from Human iPSCs. In: Tanimizu N, editor. Hepatic Stem Cells, vol. 1905, New York, NY: Springer New York; 2019, p. 131-42. https://doi.org/10.1007/978-1-4939-8961-4_12.
[19] Koui Y, Kido T, Ito T, Oyama H, Chen S-W, Katou Y, et al. An In Vitro Human Liver Model by iPSC-Derived Parenchymal and Non-parenchymal Cells. Stem Cell Reports 2017;9:490-8. https://doi.org/10.1016/j.stemcr.2017.06.010.
[20] Asai A, Aihara E, Watson C, Mourya R, Mizuochi T, Shivakumar P, et al. Paracrine signals regulate human liver organoid maturation from induced pluripotent stem cells. Development 2017; 144: 1056-64. https://doi.org/10.1242/dev. 142794.
[21] Freyer N, Greuel S, Knöspel F, Strahl N, Amini L, Jacobs F, et al. Effects of Co-Culture Media on Hepatic Differentiation of hiPSC with or without HUVEC Co-Culture. IJMS 2017; 18:1724. https://doi.org/10.3390/ijms18081724.
[22] Hwang N S, Varghese S, Elisseeff J. Controlled differentiation of stem cells. Advanced Drug Delivery Reviews 2008;60:199-214. https://doi.org/10.1016/j.addr.2007.08.036.
[23] Agarwal T, Subramanian B, Maiti T K. Liver Tissue Engineering: Challenges and Opportunities. ACS Biomater Sci Eng 2019;5:4167-82. https://doi.org/10.1021/acsbiomaterials.9b00745.
[24] Jain E, Damania A, Kumar A. Biomaterials for liver tissue engineering. Hepatol Int 2014;8:185-97. https://doi.org/10.1007/s12072-013-9503-7.
[25] Vasanthan K S, Subramanian A, Krishnan U M, Sethuraman S. Role of biomaterials, therapeutic molecules and cells for hepatic tissue engineering. Biotechnology Advances 2012;30:742-52. https://doi.org/10.1016/j.biotechadv.2012.01.004.
[26] Mohammadpour A, Arjmand S, Lotfi A S, Tavana H, Kabir-Salmani M. Promoting hepatogenic differentiation of human mesenchymal stem cells using a novel laminin-containing gelatin cryogel scaffold. Biochemical and Biophysical Research Communications 2018;507:15-21. https://doi.org/10.1016/j.bbrc.2018.10.121.
[27] Takebe T, Sekine K, Enomura M, Koike H, Kimura M, Ogaeri T, et al. Vascularized and functional human liver from an iPSC-derived organ bud transplant. Nature 2013;499:481-4. https://doi.org/10.1038/nature12271.
[28] Takebe T, Enomura M, Yoshizawa E, Kimura M, Koike H, Ueno Y, et al. Vascularized and Complex Organ Buds from Diverse Tissues via Mesenchymal Cell-Driven Condensation. Cell Stem Cell 2015; 16:556-65. https://doi.org/10.1016/j.stem.2015.03.004.
[29] Lou Y-R, Leung A W. Next generation organoids for biomedical research and applications. Biotechnology Advances 2018;36:132-49. https://doi.org/10.1016/j.biotechadv.2017.10.005.
[30] Sakabe K, Takebe T, Asai A. Organoid Medicine in Hepatology. Clinical Liver Disease 2020; 15:3-8. https://doi.org/10.1002/cld.855.
[31] Bell C C, Lauschke V M, Vorrink S U, Palmgren H, Duffin R, Andersson T B, et al. Transcriptional, Functional, and Mechanistic Comparisons of Stem Cell-Derived Hepatocytes, HepaRG Cells, and Three-Dimensional Human Hepatocyte Spheroids as Predictive In Vitro Systems for Drug-Induced Liver Injury. Drug Metab Dispos 2017;45:419-29. https://doi.org/10.1124/dmd.116.074369.
[32] Jozefczuk J, Prigione A, Chavez L, Adjaye J. Comparative analysis of human embryonic stem cell and induced pluripotent stem cell-derived hepatocyte-like cells reveals current drawbacks and possible strategies for improved differentiation. Stem Cells Dev 2011;20:1259-75. https://doi.org/10.1089/scd.2010.0361.
[33] Godoy P, Schmidt-Heck W, Natarajan K, Lucendo-Villarin B, Szkolnicka D, Asplund A, et al. Gene networks and transcription factor motifs defining the differentiation of stem cells into hepatocyte-like cells. J Hepatol 2015;63:934-42. https://doi.org/10.1016/j.jhep.2015.05.013.
[34] Gao Y, Zhang X, Zhang L, Cen J, Ni X, Liao X, et al. Distinct Gene Expression and Epigenetic Signatures in Hepatocyte-like Cells Produced by Different Strategies from the Same Donor. Stem Cell Reports 2017;9:1813-24. https://doi.org/10.1016/j.stemcr.2017.10.019.
[35] Pashos E E, Park Y, Wang X, Raghavan A, Yang W, Abbey D, et al. Large, Diverse Population Cohorts of hiPSCs and Derived Hepatocyte-like Cells Reveal Functional Genetic Variation at Blood Lipid-Associated Loci. Cell Stem Cell 2017;20:558-570.e10. https://doi.org/10.1016/j.stem.2017.03.017.
[36] Schwartz R E, Fleming H E, Khetani S R, Bhatia S N. Pluripotent stem cell-derived hepatocyte-like cells. Biotechnology Advances 2014;32:504-13. https://doi.org/10.1016/j.biotechadv.2014.01.003.
[37] Asumda F Z, Hatzistergos K E, Dykxhoorn D M, Jakubski S, Edwards J, Thomas E, et al. Differentiation of hepatocyte-like cells from human pluripotent stem cells using small molecules. Differentiation 2018; 101:16-24. https://doi.org/10.1016/j.diff.2018.03.002.
[38] Luce E, Messina A, Duclos-Vallée J, Dubart-Kupperschmitt A. Advanced techniques and awaited clinical applications for human pluripotent stem cell differentiation into hepatocytes. Hepatology 2021:hep.31705. https://doi.org/10.1002/hep.31705.
[39] Kapałczyńska M, Kolenda T, Przybyła W, Zajączkowska M, Teresiak A, Filas V, et al. 2D and 3D cell cultures - a comparison of different types of cancer cell cultures. Aoms 2016. https://doi.org/10.5114/aoms.2016.63743.
[40] Messina A, Morelli S, Forgacs G, Barbieri G, Drioli E, De Bartolo L. Self-assembly of tissue spheroids on polymeric membranes: Self-assembly of spheroids on polymeric membranes. J Tissue Eng Regen Med 2017;11:2090-103. https://doi.org/10.1002/term.2105.
[41] Wallin R, Hutson S M. Dexamethasone Stimulates Vitamin K-Dependent Carboxylase Activity in Neonatal Rats and Cultured Fetal Hepatocytes. Pediatr Res 1991;30:281-5. https://doi.org/10.1203/00006450-199109000-00017.
[42] Ren P, de Feijter A W, Paul D L, Ruch R J. Enhancement of liver cell gap junction protein expression by glucocorticoids. Carcinogenesis 1994; 15:1807-13. https://doi.org/10.1093/carcin/15.9.1807.
[43] Li Z-Q, He F-Y, Stehle C J, Wang Z, Kar S, Finn F M, et al. Vitamin K uptake in hepatocytes and hepatoma cells. Life Sciences 2002;70:2085-100. https://doi.org/10.1016/S0024-3205(01)01525-9.
[44] Avior Y, Levy G, Zimerman M, Kitsberg D, Schwartz R, Sadeh R, et al. Microbial-derived lithocholic acid and vitamin K 2 drive the metabolic maturation of pluripotent stem cells-derived and fetal hepatocytes: AVIOR ET AL. Hepatology 2015;62:265-78. https://doi.org/10.1002/hep.27803.
[45] Dihingia A, Ozah D, Ghosh S, Sarkar A, Baruah P K, Kalita J, et al. Vitamin K1 inversely correlates with glycemia and insulin resistance in patients with type 2 diabetes (T2D) and positively regulates SIRT1/AMPK pathway of glucose metabolism in liver of T2D mice and hepatocytes cultured in high glucose. The Journal of Nutritional Biochemistry 2018;52:103-14. https://doi.org/10.1016/j.jnutbio.2017.09.022.
[46] Bordoloi J, Ozah D, Bora T, Kalita J, Manna P. Gamma-glutamyl carboxylated Gas6 mediates the beneficial effect of vitamin K on lowering hyperlipidemia via regulating the AMPK/SREBP1/PPARα signaling cascade of lipid metabolism. The Journal of Nutritional Biochemistry 2019;70:174-84. https://doi.org/10.1016/j.jnutbio.2019.05.006.
[47] Jafarpour Z, Soleimani M, Hosseinkhani S, M H M H, Yaghmaei P, Mobarra N, et al. Efficient Production of Hepatocyte-like Cells from Human-induced Pluripotent Stem Cells by Optimizing Growth Factors. Int J Organ Transplant Med 2018;9:77-87.
[48] Qin J, Chang M, Wang S, Liu Z, Zhu W, Wang Y, et al. Connexin 32-mediated cell-cell communication is essential for hepatic differentiation from human embryonic stem cells. Sci Rep 2016;6:37388. https://doi.org/10.1038/srep37388.
[49] Du C, Feng Y, Qiu D, Xu Y, Pang M, Cai N, et al. Highly efficient and expedited hepatic differentiation from human pluripotent stem cells by pure small-molecule cocktails. Stem Cell Res Ther 2018;9:58. https://doi.org/10.1186/s13287-018-0794-4.
[50] Jetter, A.; Kullak-Ublick, G.A. Drugs and hepatic transporters: A review. Pharmacological Research 2019, 104234, doi:10.1016/j.phrs.2019.04.018.

1. A method for improving the differentiation of hepatoblasts into hepatocytes comprising culturing said hepatoblasts in hepatocyte medium supplemented with hepatocyte growth factor (HGF), glucocorticoid, and oncostatin M, wherein the hepatopcyte medium is used and refreshed every day, and wherein:
i) from the fourth, fifth, sixth, or seventh day until the end of culture, vitamin K is added as a supplement in the hepatopcyte medium,
ii) from the eighth or ninth day of culture until the end of the culture, glucocorticoid is administered to the culture so that its concentration is decreased by half and reverts back to its initial concentration every other day,
iii) from the ninth, tenth or eleventh day of culture until the end of the culture, an oncostatin M concentration is decreased by half every other day until its complete removal,
iv) from the ninth, tenth or eleventh day of culture until the end of the culture, a Notch inhibitor and a TGF-β receptor inhibitor are added to the culture.

2. The method for improving the differentiation of hepatoblasts into hepatocytes according to claim 1 wherein the method is performed in 2D or 3D culture.

3. The method for improving the differentiation of hepatoblasts into hepatocytes according to claim 1, wherein the glucocorticoid is dexamethasone.

4. The method for improving the differentiation of hepatoblasts into hepatocytes according to claim 1, wherein the vitamin K is vitamin K1.

5. The method for improving the differentiation of hepatoblasts into hepatocytes according to claim 1, wherein the notch inhibitor is compound E.

6. The method for improving the differentiation of hepatoblasts into hepatocytes according to claim 1, wherein the TGF-β receptor inhibitor is SB431542.

7. The method for improving the differentiation of hepatoblasts into hepatocytes according to claim 6, wherein the hepatocyte growth factor is added to the hepatopcyte medium at a concentration ranging from 10 ng/ml to 100 ng/ml, the glucocorticoid is dexamethasone and is added to the hepatopcyte medium at a concentration ranging from 0.05 nM to 0.15 nM, the oncostatin M is added to the hepatopcyte medium at a concentration ranging from 5 ng/mL to 30 ng/ml, the vitamin K is vitamin K1 and is added to the hepatopcyte medium from the seventh day of culture at a concentration ranging from 5 ng/mL to 1 mg/mL, the notch inhibitor is compound E and is added to the hepatopcyte medium from the ninth day of culture at a concentration ranging from 0.3 nM to 0.7 nM, and the TGF-β receptor inhibitor is SB431542 and is added to the hepatopcyte medium from the ninth day of culture at a concentration ranging from 3 nM to 7 nM.

8. The method for improving the differentiation of hepatoblasts into hepatocytes according to claim 7, wherein the hepatocyte growth factor is added to the hepatopcyte medium at a concentration of 20 ng/ml, the dexamethasone is added in the hepatopcyte medium at a concentration of 0.1 nM, and from the ninth day of culture the dexamethasone is administered to the hepatopcyte medium so that its concentration is decreased by 0.05 nM and reverts back to 0.1 nM every 24 h, the oncostatin M is added to the hepatopcyte medium at a concentration of 20 ng/ml, the vitamin K1 is added to the hepatopcyte medium i) from the seventh day of culture at a concentration of 10 ng/ml, the notch inhibitor is added to the hepatopcyte medium from the ninth day of culture at a concentration of 0.5 nM, the TGF-β receptor inhibitor is added to the hepatopcyte medium from the ninth day of culture at a concentration of 5 nM.

9. (canceled)

10. A method of conducting cell-based or regenerative therapy in a subject in need thereof, comprising administering to the subject a therapeutically effective amount of hepatocytes obtained by the method of claim 1.

11. (canceled)

12. A method for treating liver diseases in a subject in need thereof, comprising administering to the subject a therapeutically effective amount of hepatocytes obtained by the method of claim 1.

13. A bio-artificial liver devices comprising a population of hepatocytes obtained by the method of claim 1.

14. (canceled)

15. An in vitro method of screening for a compound useful in the treatment of liver disease and/or of conducting a toxicological study of the compound, comprising the steps of:
(a) contacting a population of hepatocytes produced by the method of claim 1 with the compound, wherein the hepatoblasts are obtained from healthy or diseased patients, and
(b) determining the effect of the compound on said hepatocytes.
